(19) United States
(12) Patent Application Publication
(10) Pub. No.: US 2014/0088768 A1
(43) Pub. Date: Mar. 27, 2014

(54) AUTOMATED SPRAY DRIER CONTROL SYSTEM

(71) Applicant: Velico Medical, Inc., Beverly, MA (US)

(72) Inventors: Michael Haley, Marlboro, MA (US); Dennis Brian Hubbard, JR., Lancaster, MA (US); Samira Johnson, Beverly, MA (US); Abdul Wahid Khan, Murrysville, PA (US)

(21) Appl. No.: 13/953,458

(22) Filed: Jul. 29, 2013

Related U.S. Application Data
(60) Provisional application No. 61/706,759, filed on Sep. 27, 2012.
(60) Provisional application No. 61/820,428, filed on May 7, 2013.
(60) Provisional application No. 61/856,954, filed on Jul. 22, 2013.

(51) Int. Cl. A61M 1/38; G05D 16/00

(52) U.S. Cl. CPC A61M 1/38; G05D 16/00
USPC 700/283

(57) ABSTRACT

A spray drier monitoring and control system is provided for a spray drier device adapted to spray dry a liquid sample such as blood plasma. The system may receive information regarding an operator of the device, the liquid sample, a spray drier assembly used by the device for spray drying, and spray drying process parameters. The spray drier system may also conduct a plurality of checks to ensure that traceability and conformance with attendant quality/safety standards are maintained throughout the spray drying process. The information may be output by the system to a remote computing device (e.g., a blood center computing system) that tracks the sample history. Beneficially, the spray drier system allows information regarding spray drying to be easily integrated with the sample information for enhanced tracking and quality control of the sample.

### RELATED APPLICATION(S)

This application claims the benefit of priority of U.S. Provisional Patent Application No. 61/706,759, filed on Sep. 27, 2012 and entitled, “Automated Spray Drier System,” U.S. Provisional Patent Application No. 61/820,428, filed on May 7, 2013 and entitled, “Functionally Closed System Equivalence For Aerosoling And Drying Gas,” and U.S. Provisional Patent Application No. 61/856,954, filed on Jul. 22, 2013 and entitled “Automated Spray Drier,” the entirety of each of which is hereby incorporated by reference.

### GOVERNMENT SUPPORT

This invention was made with Government support under contract HHSO100201200005C awarded by the Biomedical Advanced Research and Development Authority (BARDA). The Government has certain rights in the invention.

### BACKGROUND

Making up about 55% of the total volume of whole blood, blood plasma is a whole blood component which holds blood cells in suspension. Blood plasma further contains a mixture of over 700 proteins and additional substances that perform functions necessary for bodily health, including clotting, protein storage, and electrolytic balance, amongst others. When extracted from whole blood, blood plasma may be employed to replace bodily fluids, antibodies, and clotting factors. Accordingly, blood plasma is extensively used in medical treatments.

To facilitate storage and transportation of blood plasma until use, plasma is typically preserved by fresh-freezing. Fresh-Frozen blood Plasma (FFP) is obtained through a series of steps involving centrifugation of whole blood to separate plasma and then freezing the collected plasma within about 8 hours of drawing the whole blood. In the United States, the American Association of Blood Banks (AABB) standard for FFP is up to 12 months from the date of preparation when stored at −18° C. or colder. FFP may also be stored for up to 7 years if maintained at −65° C. or colder from preparation until the time at which it used. In Europe, FFP has a shelf life of only 3 months if stored at temperatures between −18° C. to −25° C., and for up to 36 months if stored at colder than −25° C. If thawed, European standards dictate that the plasma must be transfused immediately or stored at 1° C. to 6° C. and transfused within 24 hours. If stored longer than 24 hours, the plasma must be relabeled for other uses or discarded.

Notably, however, FFP must be kept within a temperature-controlled environment throughout its duration of storage to maintain its efficacy, which adds to the cost and difficulty of storage and transport. Furthermore, FFP must be thawed prior to use, resulting in a delay of 30-45 minutes before it may be used after removal from cold storage.

Accordingly, there is a need to develop alternative techniques for storage of plasma.

### SUMMARY

In an embodiment, a tracking system is provided for tracking a liquid sample undergoing spray drying operations by a spray drier device. The tracking system may include a first computing device in communication with a spray drier device. The first computing device may be adapted to receive operator information regarding an operator of a spray drier device, sample information regarding a liquid sample to be spray dried by the spray drier device, assembly information regarding a spray drier assembly adapted to couple with the spray drier device for spray drying the liquid sample, and operating parameters for a spray drying operation performed by the spray device to dry the liquid sample. The tracking system may further include a user interface in communication with the first computing device, the user interface adapted to display at least one status associated with one or more of the operator information, the sample information, the assembly information, and the operating parameters. The first computing device may be further adapted to output at least one of the operator information, the sample information, the assembly information, and the operating parameters to a second computing device maintaining a record of the sample information.

In a further embodiment of the tracking system, the spray drier device may include a liquid sample port for receiving a flow of a liquid sample and a spray drier device dock adapted to couple with the spray drier assembly positioned within the dock. The dock may include an aerosolizing gas port for receiving the flow of an aerosolizing gas and a dryer gas port for receiving the flow of drying gas. The aerosolizing gas port may not be co-axial with the dryer gas port.

In a embodiment of the system, the spray drier device may also include a locking mechanism positioned adjacent to the dock and configured to couple with a spray drier assembly positioned within the dock and an actuator configured to move the locking mechanism between an engaged and a disengaged position. The locking mechanism may inhibit removal of the spray drier assembly from the dock in the engaged position and the locking mechanism may not inhibit removal of the spray drier assembly from the dock in the disengaged position.

In an embodiment of the tracking system, the operator information may include an identity of the operator.

In an embodiment of the tracking system, the sample information may include one or more of a donor identification number (DIN), a sample collection date, a sample volume, and a sample expiration date.

In an embodiment of the tracking system, the assembly information may include one or more of an assembly product code, an assembly lot number, and an assembly expiration date.

In an embodiment of the tracking system, the operating parameters may include one or more of flow rate and temperature for one or more of the flows of liquid sample, aerosolizing gas, and drying gas.

In an embodiment of the tracking system, the spray drier device may further include a plurality of heaters adapted to heat the spray drier assembly by electromagnetic radiation, where the operating parameters include output of the plurality of heaters.

In an embodiment of the tracking system, the first computing device is adapted to receive one or more of the operator information, the sample information, and the assembly information by bar code.

In an embodiment of the tracking system, the first computing device is further adapted to compare the operating parameters to a plurality of quality control criteria and determine a quality of the spray drying operation based upon the comparison.

In an embodiment of the tracking system, the first computing device is adapted for remote access by an operator for performing one or more of monitoring the operating parameters during the spray drying operation, reviewing spray drier device alerts, and performing diagnostics on the spray drier device.

In an embodiment of the tracking system, the first computing device is in communication with at least two spray drier devices, where the first computing device is adapted to receive a plurality of status indications from each of the spray drier devices and display the respective status indications for each of the spray drier devices on a common display device.

In an embodiment of the tracking system, the first computing device is in communication with a data store, the data store adapted to maintain operating parameters for a plurality of spray drying operations performed by the spray drier device.

In an embodiment, a method for tracking a liquid sample undergoing spray drying is provided. The method may include receiving, by a first computing device in communication with a spray drier device operator information regarding an operator of a spray drier device, sample information regarding a liquid sample to be spray dried by the spray drier device, assembly information regarding a spray drier assembly adapted to couple with the spray drier device for spray drying the liquid sample, and operating parameters for a spray drying operation performed by the spray device to dry the liquid sample. The method may further include displaying, by a user interface in communication with the first computing device, at least one status associated with one or more of the operator information, the sample information, the assembly information, and the operating parameters. The method may additionally include outputting, by the first computing device, at least one of the operator information, the sample information, the assembly information, and the operating parameters to a second computing device maintaining a record of the sample information.

In an embodiment of the method, the spray drier device includes a liquid sample port for receiving the flow of a liquid sample and a spray drier device dock adapted to couple with the spray drier assembly positioned within the dock. The dock may include an aerosolizing gas port for receiving the flow of an aerosolizing gas and a dryer gas port for receiving the flow of drying gas. In certain embodiments, the aerosolizing gas port may not be co-axial with the dryer gas port.

In an embodiment of the method, the spray drier device may further include a locking mechanism positioned adjacent to the dock and configured to couple with a spray drier assembly positioned within the dock and an actuator configured to move the locking mechanism between an engaged and a disengaged position. The locking mechanism may inhibit removal of the spray drier assembly from the dock in the engaged position and the locking mechanism may not inhibit removal of the spray drier assembly from the dock in the disengaged position.

In an embodiment of the method, the operator information may include an identity of the operator.

In an embodiment of the method, the sample information may include one or more of a donor identification number (DIN), a sample collection date, a sample volume, and a sample expiration date.

In an embodiment of the method, the assembly information may include one or more of an assembly product code, an assembly lot number, and an assembly expiration date.

In an embodiment of the method, the operating parameters may include one or more of flow rate and temperature for one or more of the flows of liquid sample, aerosolizing gas, and drying gas.

In an embodiment of the method, the spray drier device may further include a plurality of heaters adapted to heat the spray drier assembly by electromagnetic radiation and where the operating parameters include output of the plurality of heaters.

In an embodiment, the method may further include comparing the operating parameters to a plurality of quality control criteria and determining a quality of the spray drying operation based upon the comparison.

In an embodiment the method may further include providing, by the first computing device, remote access to the spray drier device by an operator of the spray drier device for performing one or more of monitoring the operating parameters during the spray drying operation, reviewing spray drier device alerts, and performing diagnostics on the spray drier device

In an embodiment, the method may further include receiving, by the first computing device, a plurality of status indications from each of the spray drier devices and displaying, by the first computing device, the respective status indications for each of the spray drier devices on a common display device.

In an embodiment, the method may also include communicating, by the first computing device, with a data store, the data store adapted to maintain operating parameters for a plurality of spray drying operations performed by the spray drier device.

In an embodiment of the method, the first computing device may be adapted to receive one or more of the operator information, the sample information, and the assembly information by bar code.

In an embodiment, a method of controlling a spray drying operation is provided for producing a spray dried sample from a liquid sample. The method may include receiving, by a first computing device, sample information regarding a liquid sample to be spray dried by a spray drier device, and assembly information regarding a spray drier assembly adapted to couple with the spray drier device for spray drying the liquid sample. The method may also include comparing, by the first computing device, each of the sample information and the assembly information to a plurality of respective reference values. The method may also include authorizing, by the first computing device, the spray drier device to perform a spray drying operation to spray dry the liquid sample for liquid sample information and assembly information satisfying their respective plurality of reference values.

In an embodiment, the method may further include not authorizing the spray drier device to spray dry the liquid sample for either liquid sample information or assembly information failing to satisfy their respective plurality of reference values

In an embodiment of the method, the liquid sample information and the spray drier assembly information may each include respective expiration dates and the respective reference values each include a current date for spray drying operations, where the liquid sample information and the spray drier assembly information each satisfy their respective reference value for current dates prior to the respective expiration dates.

In an embodiment of the method, the liquid sample information may include a volume of the liquid sample and the plurality of reference values for the liquid sample may include a volume of the spray drier assembly. The liquid sample information may satisfy the plurality of reference values for the liquid sample for liquid sample volumes less than the spray drier assembly volume.

In an embodiment of the method, comparing each of the sample information and the assembly information to a plurality of respective reference values may further include printing, by a printing device in communication with the first computing device, a bar code including the sample information, affixing the bar code including the sample information and the assembly information to the spray drier assembly, and comparing the sample information and the assembly information included in the bar code to the plurality of respective reference values.

In an embodiment, the method may further include outputting, by the first computing device, at least one of the operator information, the sample information, the assembly information, and operating parameters for the authorized spray drying operation to a second computing device maintaining a record of the sample information

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1A is a schematic illustration of an embodiment of a spray drier system of the present disclosure, including a spray drier device and a spray drier assembly;

FIG. 1B is a schematic illustration of a plurality of the spray drier systems of FIG. 1 for use with a pooled liquid source;

FIGS. 2A and 2B are schematic illustrations of a spray drier assembly detailing embodiments of the spray drier assembly of FIG. 1A;

FIG. 2C is a perspective view of an embodiment of the spray drier assembly;

FIG. 2D is a schematic illustration of an embodiment of a collection chamber of the spray drier assembly of FIG. 1A;

FIGS. 3A-3C are views of embodiments of a head of the spray drier assembly of FIG. 1A; (A) front perspective view; (B) rear perspective view; (C) schematic, cut-away view;

FIGS. 4A-4B are schematic illustrations of embodiments of a filter support of the spray drier assembly head; (A) radially extending fins; (B) angled fins;

FIGS. 4C-4D are perspective, three-dimensional views of embodiments of the filter support of FIGS. 4A-4B;

FIGS. 5A-5B are schematic illustrations of embodiments of the spray drier system of FIG. 1A, illustrating gas flow pathways between the spray drier device and spray drier assembly;

FIGS. 6A-6B are perspective views of an embodiment of a dock of the spray drier system of FIG. 1A; (A) front view; (B) rear view;

FIG. 6C is a schematic illustration of an actuator in communication with the dock of FIGS. 6A-6B;

FIGS. 7A-7B are a perspective views of embodiments of the dock of FIGS. 6A-6B with the locking mechanism in the (A) open position and (B) closed position;

FIGS. 8A-8B are perspective views of an embodiment of a cover for the spray drier assembly of the present disclosure; (A) open position; (B) closed position;

FIG. 9 is a schematic illustration of a sealing device for sealing the collection chamber of FIG. 2D;

FIG. 10 is a flow diagram illustrating an embodiment of a workflow process for tracking the liquid plasma source and spray drier assembly employed in a spray drying operation by the spray drier device of FIGS. 1A-1B.

FIG. 11 is a flow diagram illustrating an embodiment of a workflow process for monitoring spray drying parameters and transmitting spray drying information to a remote computing device; and

FIG. 12 is a schematic illustration of an embodiment of a user interface of the spray drier device of FIGS. 1A-1B.

### DETAILED DESCRIPTION

Embodiments of the present disclosure are directed to systems and methods for spray drying a liquid sample. In certain embodiments, the liquid sample is plasma obtained from a blood donor. However, it may be understood that embodiments of the disclosed spray drier systems and methods may be employed to spray dry any mixtures of solid particles in a continuous liquid medium, including, but not limited to, colloids, suspensions, and sols.

In general, a spray drier system is provided for spray drying a liquid sample such as blood plasma. In an embodiment, the spray drier system of the present technology includes a spray drier device and a spray drier assembly. The spray drier device is configured, in an aspect, to receive flows of an aerosolizing gas, a drying gas, and a plasma from respective sources and reversibly couple with the spray drier assembly for transmission of the received aerosolizing gas, drying gas, and plasma to the spray drier assembly. Spray drying of the plasma is performed in the spray drier assembly under the control of the spray drier device.

In certain embodiments, the spray drier assembly includes a spray drying head, a drying chamber, and a collection chamber. During spray drying, the flow of blood plasma, aerosolizing gas, and drying gas may be received at the spray drier head. Within the spray drier head, in an embodiment, the blood plasma is aerosolized using the aerosolizing gas to form an aerosolized blood plasma. The aerosolized plasma may be further mixed with the drying gas in the spray drier head and emitted into the drying chamber. In the drying chamber, contact between the aerosolized plasma and the drying gas causes moisture to move from the aerosolized plasma to the drying gas, producing dried plasma and humid drying gas. In this embodiment, the dried plasma and humid drying gas subsequently flow into the collection chamber, where the dried plasma is isolated from the humid drying gas and collected, while the humid drying gas is exhausted from the spray drier assembly into the device for recirculation (e.g., a closed system), or into the outside air, as further described herein (e.g., an open system).

In alternative embodiments, the aerosolizing gas may be omitted and the spray drier assembly head may include an aerosolizer that receives and atomizes the flow of plasma. Examples of the aerosolizer may include, but are not limited to, ultrasonic atomizing transducers, ultrasonic humidified transducers, and piezo-ultrasonic atomizers.

The spray drier device may further include one or more conditioners devices adapted to modify one or more properties of the plasma, aerosolizing gas, and/or drying gas. Such properties may include, but are not limited to, temperature, pressure, moisture content, purity (e.g., bacterial load/contamination), and flow rate. Conditioner examples include, but are not limited to, heaters, pumps, filters, dehumidifiers, humidifiers, regulators, valves, and like.

The spray drier assembly and spray drier device may optionally include a plurality of secondary heaters that, in combination with the drying air, assist in drying the aerosolized plasma. In one embodiment, a heater may be positioned at or near the point of aerosolization or shortly thereafter in the path of the aerosolized plasma (e.g., in the drying chamber). In another embodiment, the secondary heater may be adapted to irradiate the spray drier assembly 104. For example, the secondary heater may be positioned outside the spray drier assembly 104 and energy (e.g., electromagnetic, radio-frequency, radiation, microwaves, etc.) is directed through the wall of the spray drier assembly for heating (e.g., heating the drying chamber 104B and/or heating the collection chamber 104C).

Providing secondary heaters may provide additional benefits beyond just temperature control. For example, by providing secondary heaters for heating of the drying chamber, the temperature of the flow of drying gas entering the spray drier assembly may be reduced. That is to say, the temperature of the flow of drying gas entering the spray drier assembly does not need to be elevated in order to account for heat loss within the spray drier assembly. Accordingly, the filter may be rated to operate at a lower temperature, allowing the use of cheaper filter, which reduces the cost of the spray drier assembly.

After collecting the dried plasma within the collection chamber, the collection chamber can be separated from the spray drier assembly and hermetically sealed. In this manner, the sealed collection chamber sterilely stores the dried plasma until use. In a certain embodiment, the collection chamber includes a plurality of ports allowing a rehydration solution to be placed in fluid communication with the collection chamber. Flow of the rehydration solution into the collection chamber reconstitutes the plasma for use in treating an individual.

In an embodiment of the present technology, a collection chamber may include a separate second vessel or container for storing/maintaining the rehydration solution. A seal is further present between the dried plasma and the rehydration solution. When reconstitution of the dried plasma is desired, the second vessel is brought into fluid communication with the dried plasma (e.g., the user breaks the seal to allow communication or contact between the rehydration solution and the dried plasma).

In certain embodiments, the spray drier device may further include a dock. The dock, in an embodiment, is configured to receive flows of both the aerosolizing gas and drying gas from their respective sources via a plurality of conduits. In this embodiment, the dock is configured to sterilely engage with the head of the spray drying assembly for transmission of the aerosolizing gas and drying gas from the spray drier device to the spray drier assembly. In certain embodiments, the flow of liquid sample is also received at the dock and transmitted to the spray drier assembly. In other embodiments, the flow of liquid sample is received directly at the spray drier assembly, without passing through the dock.

The spray drier assembly head may further include quick-connect ports for receiving the drying gas and the aerosolizing gas (and optionally, the liquid sample, when the liquid sample is directed to the dock). Beneficially, this confirmation may reduce the number of operations an operator must perform to connect the spray drier assembly to the spray drier device, allowing for more ergonomic and quicker processing of blood, as well as fewer errors. Additionally, by avoiding transmission of the gas (and optionally liquid) flows to the spray drier assembly through the walls of the spray drier assembly body, sterile integrity of the spray drier assembly may be more easily maintained. Furthermore, eliminating multi-piece connections may reduce the complexity and cost of the spray drier assembly.

Embodiments of the dock may also include a locking mechanism configured to move between a closed position and an open position. In the open position, the head of the spray drier assembly may be freely positioned within the dock or removed from the dock. In the closed position, a spray drier assembly head positioned within the dock is prohibited from being removed from the dock by the locking mechanism.

By providing a spray drier assembly that can be removed from the spray drier device, the spray drier assembly can be configured for use multiple times or used a single time. In each case, the spray drier head, spray drying chamber, and collection chamber are each made from materials that are sterile or can be sterilized. In an embodiment, sterilization may be performed through various techniques including, but not limited to, autoclave sterilization, light sterilization, radiation sterilization, heat sterilization, chemical/gas sterilization, pressure sterilization, and a combination thereof. In another embodiment, pieces of the spray dry system or assembly may be formed from, or coated with, materials that resist or minimize bacterial, fungal or viral growth (e.g., materials impregnated with or made from silver, copper, chlorhexidine, antibiotics, and the like).

Reusable components of the spray drier assembly are sterilized prior to each spray drying operation. For example, reusable components may include one or more of the spray drier head and drying chamber. Notably, in certain embodiments, as the collection chamber stores the dried plasma, it may be formed from disposable materials and employed in combination with the reusable components of the spray drier assembly. Each reusable component may be independently formed from materials having relatively high durability in order to withstand repeated sterilization without experiencing damage (e.g., wear). Examples may include, but are not limited to, metals, metal alloys, stainless steels, and the like.

Disposable components of the spray drier assembly are sterilized prior to spray drying operation and discarded after use. For example, disposable components may include one or more of the spray drier head, spray drying chamber, and collection chamber. Accordingly, each disposable component may be independently formed from materials having durability sufficient for sterilization prior to use, without necessarily possessing additional durability to withstand repeated sterilization. Examples may include, but are not limited to, polymers.

The use of a combination of reusable and disposable components within the spray drying assembly may provide efficiency and cost savings. For example, by providing a spray drier assembly having a reusable spray drying head and drying chamber with a single-use, disposable collection chamber, the collection chamber may be decoupled from the remainder of the assembly. Thus, a significant fraction of the spray drier assembly does not require replacement during each use, reducing the spray drying cost.

When handling transfusion products, such as plasma, the transfusion products must be maintained in a functionally closed environment from the time they are collected to the time they are transfused. In other words, collected blood components are not to be exposed to any contaminants during collection, drying, storage, and transfusion. Accordingly, embodiments of the system are further configured to provide a functionally closed environment which provides an environment that is essentially free of contaminants e.g., that contamination is at an acceptable level within the spray drying system, including in the spray drier device, spray drier assembly, or collection chamber of the present technology during spray drying operations.

The phrases “free of contaminants,” “essentially free of contaminants” and “sterile” refer to an environment, device and/or assembly that have a selected bacterial load, selected combined bacterial efficiency (BFE), or any combination thereof. In an embodiment, the bacterial load and BFE may be selected to provide a medically acceptable level of bacteria within the system. In a further non-limiting embodiment, the selected bacterial load may be approximately 1 CFU/m³ or less. In another non-limiting embodiment, the selected BFE may be 10⁶ or greater.

Each of the aerosolizing gas and drying gas within the spray drying assembly is considered sterile and meets these requirements. For example, in certain embodiments, the flows of aerosolizing gas and drying gas are passed through a plurality of filters. In this manner, gas for aerosolizing, drying, or both are introduced to the spray drying assembly essentially free of contaminants. Examples of such filters include HEPA and ULPA filters (see Table 1 below) that achieve the level of bacterial efficiency/bacterial load described herein. In alternative embodiments, other filters known in the art or developed in the future can be used so long as the bacterial efficiency/bacterial load described herein is achieved. Filters can also be layered to achieve the efficiency described above. For example, a single 0.2 micron filter can be used or, alternatively, two or more lower-efficiency filters can be placed in series to achieve the desired level of filtration efficiency.

TABLE 1
Filter Efficiency

| EN1822 | | |
|---|---|---|
| Classification of the Filter | % Clean Air | |
| | HEPA Filters Stopping Particles ≧0.3 μm | |
| H10 | 95% | Clean Air |
| H11 | 98% | Clean Air |
| H12 | 99.99% | Clean Air |
| H13 | 99.997 | Clean Air |
| H14 | 99.999% | Clean Air |
| | ULPA Filters Stopping Particles ≧0.3 μm | |
| U15 | 99.9995% | Clean Air |
| U16 | 99.99995% | Clean Air |
| U17 | 99.999995% | Clean Air |

TABLE 2
ISO 14644-1 Cleanroom Classifications

| | Maximum particles/m³ | | | | | | FED STD 209E |
|---|---|---|---|---|---|---|---|
| Class | ≧0.1 μm | ≧0.2 μm | ≧0.3 μm | ≧0.5 μm | ≧1 μm | ≧5 μm | equivalent |
| ISO 1 | 10 | 2.37 | 1.02 | 0.35 | 0.083 | 0.0029 | |
| ISO 2 | 100 | 23.7 | 10.2 | 3.5 | 0.83 | 0.029 | |
| ISO 3 | 1,000 | 237 | 102 | 35 | 8.3 | 0.29 | Class 1 |
| ISO 4 | 10,000 | 2,370 | 1,020 | 352 | 83 | 2.9 | Class 10 |
| ISO 5 | 100,000 | 23,700 | 10,200 | 3,520 | 832 | 29 | Class 100 |
| ISO 6 | 1.0 × 10⁶ | 237,000 | 102,000 | 35,200 | 8,320 | 293 | Class 1,000 |
| ISO 7 | 1.0 × 10⁷ | 2.37 × 10⁶ | 1,020,000 | 352,000 | 83,200 | 2,930 | Class 10,000 |
| ISO 8 | 1.0 × 10⁸ | 2.37 × 10⁷ | 1.02 × 10⁷ | 3,520,000 | 832,000 | 29,300 | Class 100,000 |
| ISO 9 | 1.0 × 10⁹ | 2.37 × 10⁸ | 1.02 × 10⁸ | 35,200,000 | 8,320,000 | 293,000 | Room air |

TABLE 3
Air Classifications

| GMP | A | B | C | D |
|---|---|---|---|---|
| ISO 14644-1 | 5 | 5/6 | 7 | 8 |
| FED STD | 100 | 100/1000 | 10,000 | 100,000 |
| 209E | | | | |
| Air Flow | Laminar flow at | Turbulent air | Turbulent air | Turbulent air flow |
| | working area | flow | flow | |
| Colony | 1 | 10 | 100 | 200 |
| Forming Units | | | | |
| (per m³) | | | | |
| Particles per | ≧0.5 μm: 3,500 | ≧0.5 μm: 3,500 | ≧0.5 μm: | ≧0.5 μm: |
| m³ when in | ≧5.0 μm: none | ≧5.0 μm: none | 350,000 | 3,500,000 |
| rest | | | ≧5.0 μm: 2,000 | ≧5.0 μm: 20,000 |

In other embodiments, the spray drier device includes a housing. The housing may include a ceiling and two or more walls for housing the spray drying assembly. For example, the housing may define an enclosed cavity including the dock for receiving the spray drying assembly. A unidirectional flow of filtered air may be directed from a portion of housing to (e.g., a top portion or ceiling positioned above the spray drier assembly) towards areas at and adjacent to the spray drier assembly, the dock, and the connection there between. For example, the housing may include a fan coupled with at least one filter (e.g., an environmental chamber air filter) to provide a laminar air flow incident upon areas at and adjacent to the spray drier assembly, the dock, and the connection there. This laminar air flow may be provided during the spray dry cycle as well as between spray drying cycles (e.g., during the time the spray drying assembly is connected to the dock of the spray drying device).

Beneficially, the housing and airflow may isolate the spray drier assembly from the environment outside the spray drier device. For example, the housing provides a physical barrier that inhibits contaminants from contacting the spray drier assembly. Furthermore, the provided air flow helps to prevent undesired amounts of bacteria, fungi and viron particles from build up on or near the surfaces of the spray drying device and/or the spray drier assembly. For example, this laminar flow of filtered air can be configured to provide an ISO 8 equivalent environment or better, or an environmental bacterial load of approximately 200 CFU/m³ or less. Furthermore, the positive pressure afforded by the unidirectional flow of air inhibits contaminants from entering the clean surroundings of the docking area. Additionally, the housing may provide protection for an operator in the event that a spray drier assembly ruptures under internal pressure during spray drying operations.

In further embodiments, the spray drier device may include a spray drier computing device that receives a variety of information regarding spray drying operations. The information may include, but is not limited to, information regarding an operator of the spray drier device, liquid sample information, spray drier assembly information, and spray drying process information. At least a portion of this information may be cross-checked during use of the spray drier device to ensure that the liquid sample is properly tracked during spray drying operations.

This information may further be transmitted to a remote computing device. For example, the remote computing device may be part of an inventory control system of a blood back. The remote computing device may further maintain original records regarding a plasma sample while stored in its liquid state (e.g., FFP). Once the plasma sample is spray dried, the computing system may transmit the collected spray drier information to the blood bank (e.g., remote computing device), where the blood bank's sample records are updated. Subsequently, when the dried plasma is used, the blood bank possesses a complete and accurate tracking history of the plasma sample which is readily accessible from the data structure by the remote computing device.

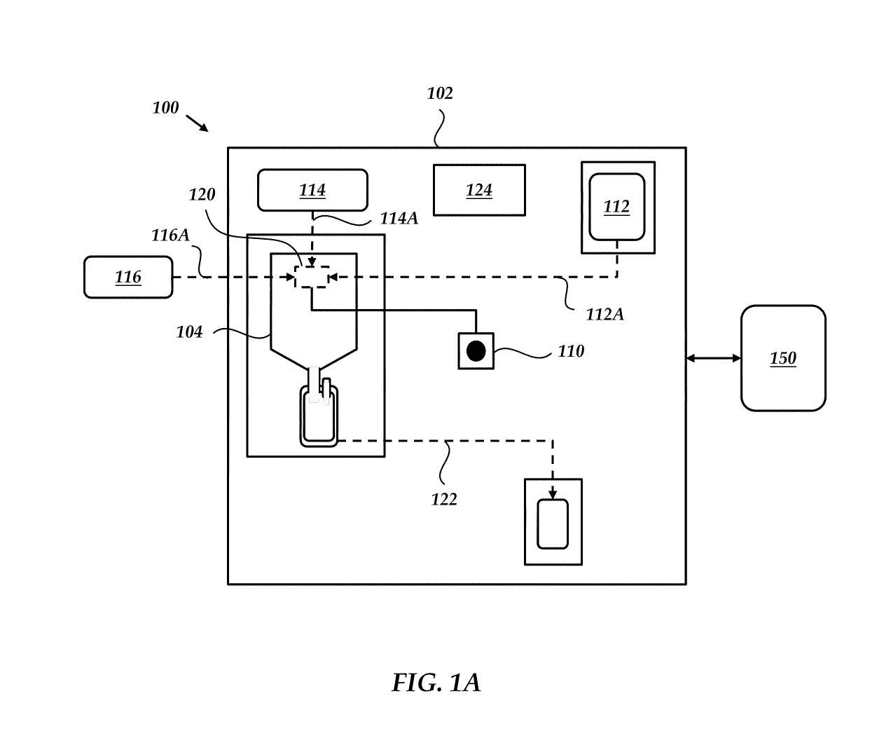

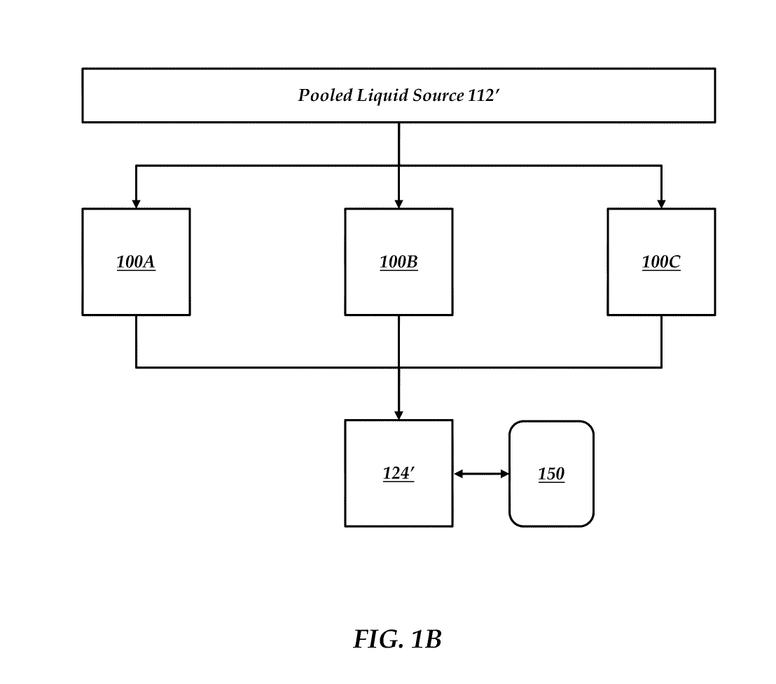

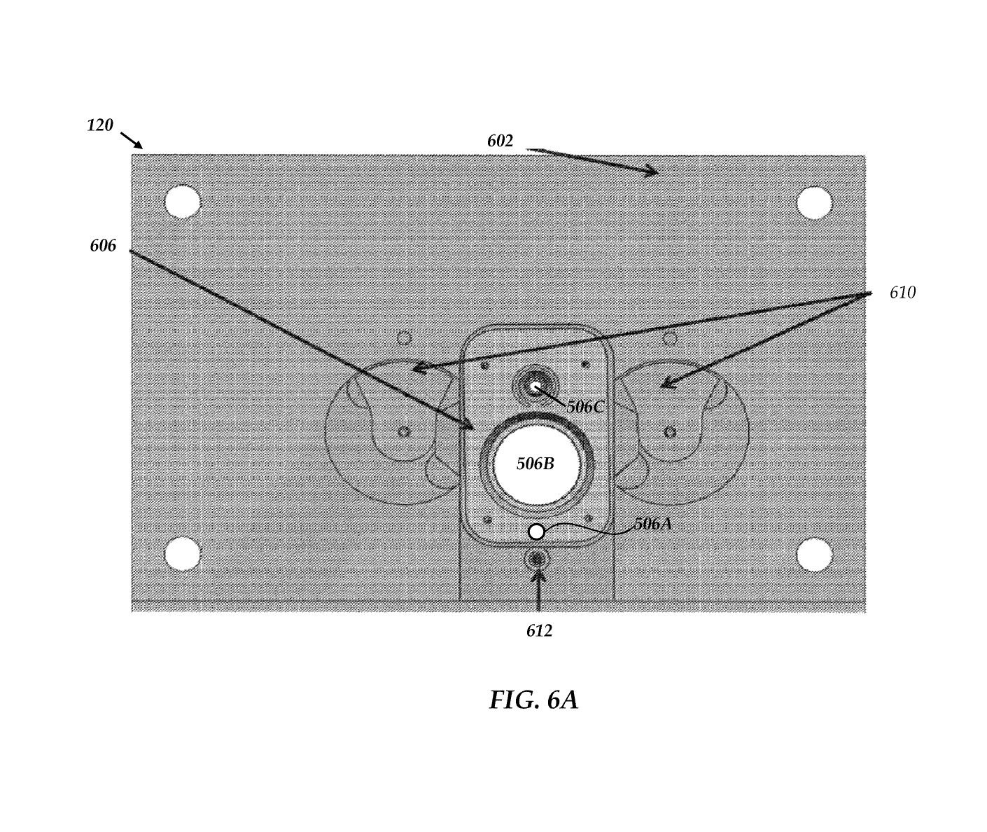

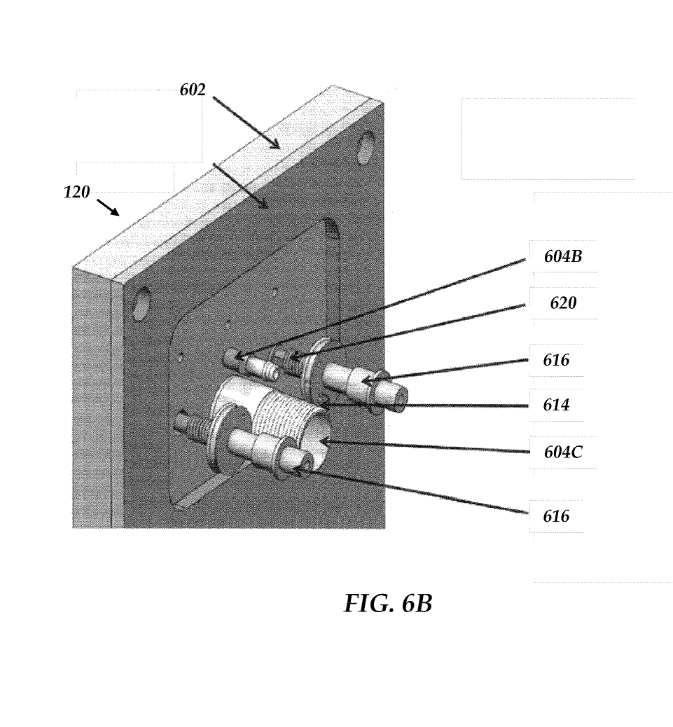

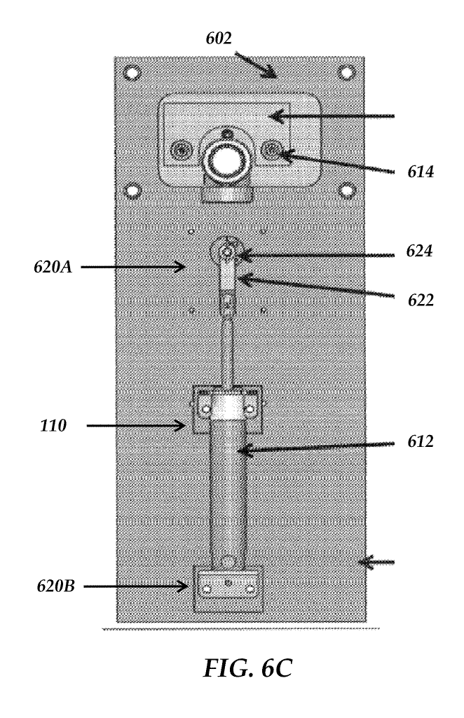

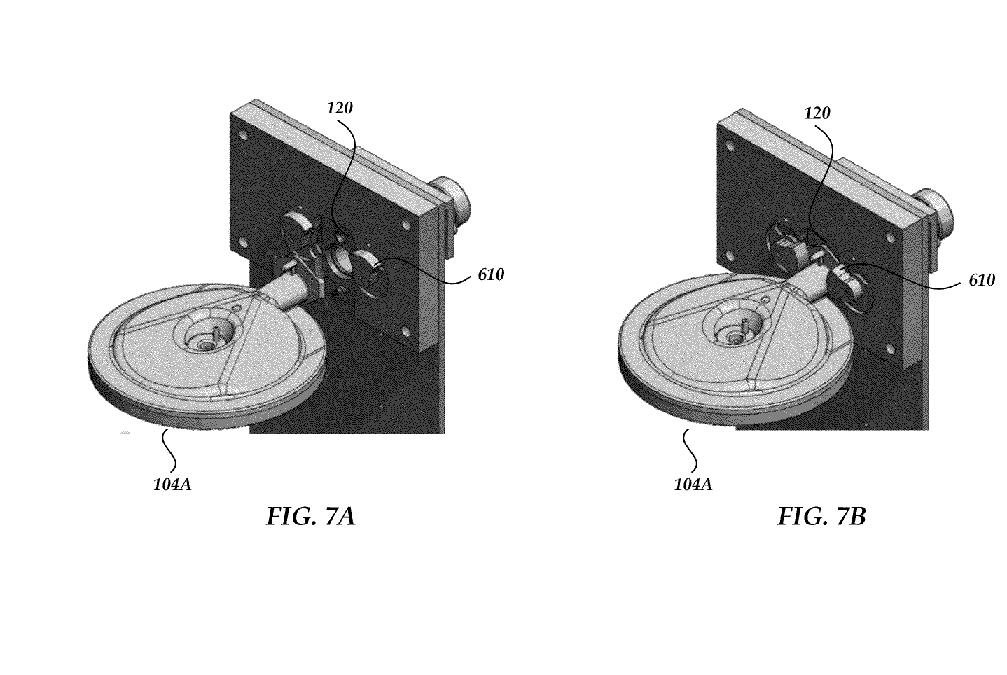

Reference will now be made to FIG. 1A, which schematically illustrates one embodiment of a spray drier system 100. In this embodiment, the system 100 includes a spray drier device 102 configured to receive a spray drier assembly 104 at a dock 120. As discussed in detail below with respect to FIGS. 6A-6B, 7A-7B, the dock 120 can further include a locking mechanism 610 in communication with an actuator 110. The actuator 110 may be employed to cause the locking mechanism 610 to engage or disengage a spray drier assembly 104 positioned within the dock 120. When the locking mechanism 610 is engaged, the spray drier assembly 104 is hermetically and sterilely sealed to the dock 120 for conducting spray drying operations. When the locking mechanism 610 is disengaged, the spray drier assembly 104 may be removed from the dock 120 for disposal or sterilization.

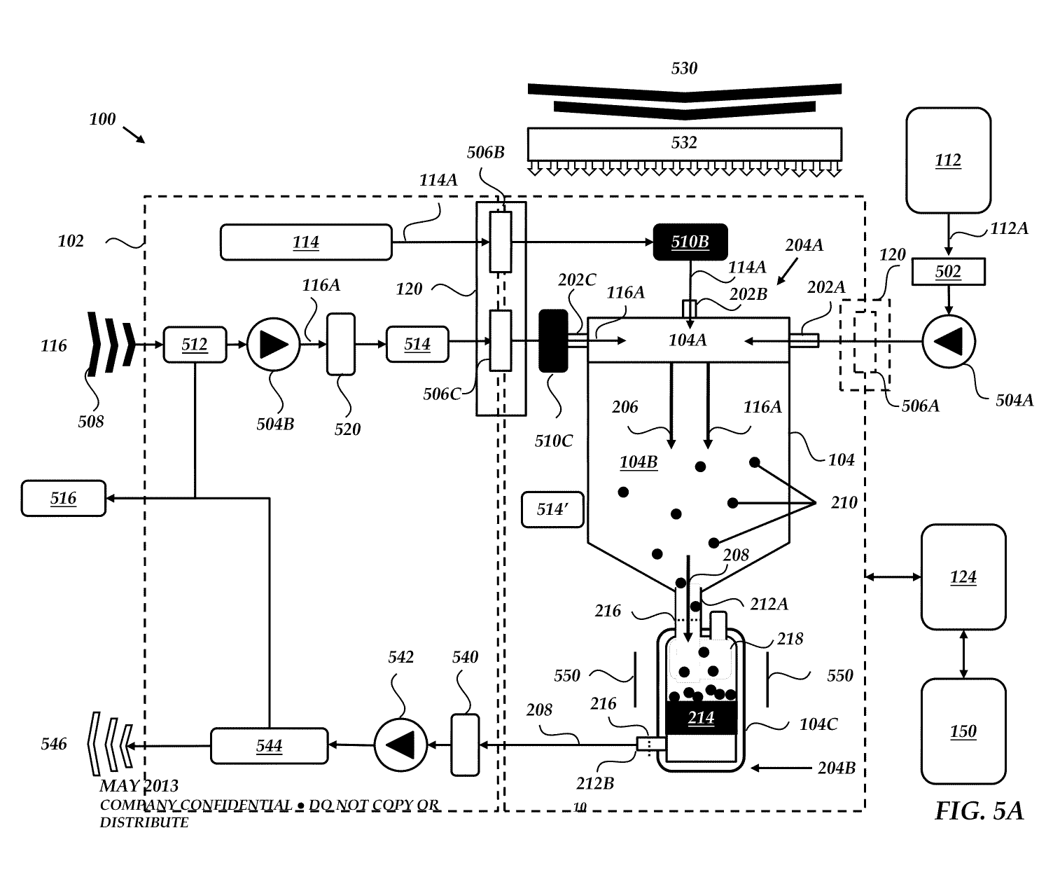

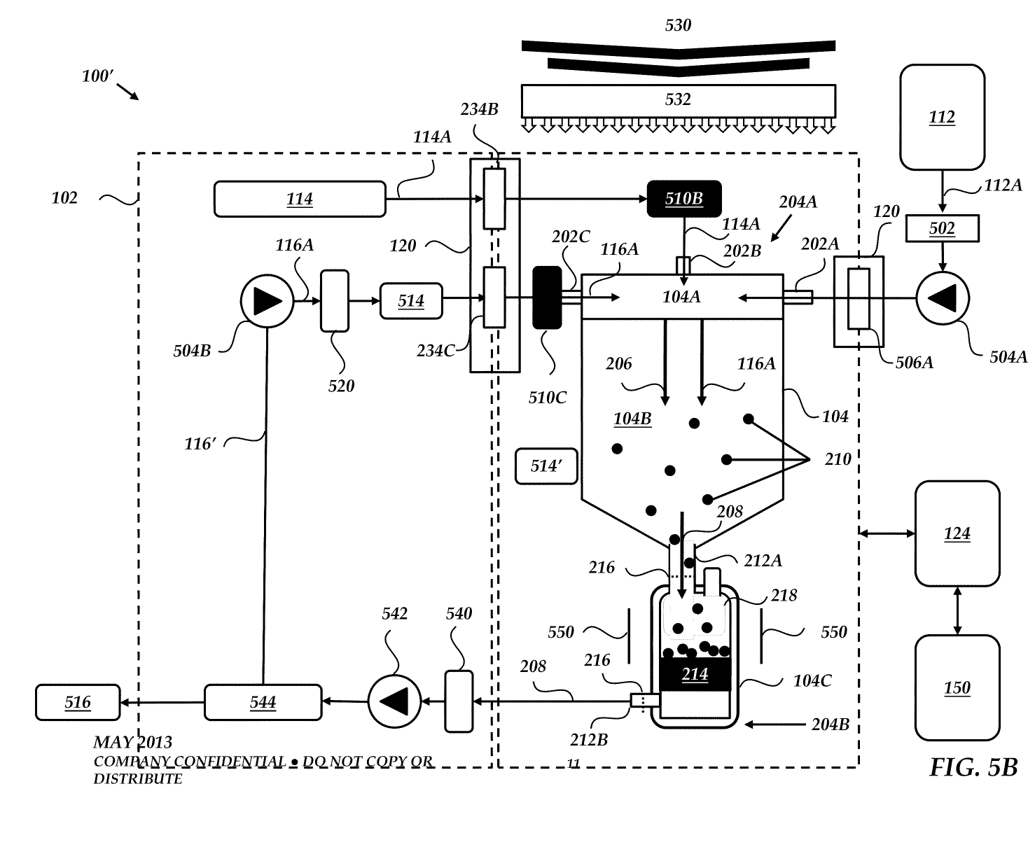

In the embodiment shown in FIG. 1A, a source of plasma 112, a source of aerosolizing gas 114, and a source of drying gas 116 are further in fluid communication with the dock 120. During spray drying operations, flows of the aerosolizing gas 114A and drying gas 116A are drawn through the spray drier device 102 at selected, respective rates, to the dock 120. As discussed in greater detail below with respect to FIGS. 5A-B, the spray drier device 102 may include conditioners (e.g., heaters, pumps, humidifiers/dehumidifiers, etc.) for altering one or more properties of the flow of drying gas 116A. For example, the flow of drying gas 116A may be heated to a temperature between about 50° C. and about 150° C., and urged to move at a flow rate of between about 15 CFM to about 35 CFM. The flow of aerosolizing gas 116A can be urged to move at a flow rate of between about 5 L/min and about 20 L/min and be heated to a temperature between about 15° C. to about 30° C. (e.g., 24° C.). The flow of liquid sample 112A may be urged to move at a flow rate of between about 3 ml/min to about 20 ml/min. The flow of the aerosolizing gas 114A, the flow of drying gas 116C, or both direct the flow of the dried sample through at least a portion of the spray drier assembly 104 (e.g., the drying chamber, the collection chamber, or both).

The spray drier assembly 104 shown in FIG. 1A is further connected to the dock 120, where the flows of the aerosolizing gas 114A and dryer gas 116A are transmitted to the spray drier assembly 104 via the dock 120. The flow of liquid sample 112A can enter the spray drier assembly 104 through the dock 120 or bypass the dock 120 and enter a spray drying head of the assembly 104 directly. In the embodiment shown in FIG. 1A, the flow of plasma 112A is further received by the spray drier assembly 104 via the dock 120. In alternative embodiments, the flow of plasma 112A is provided directly to the spray drier assembly 104 without passing through the dock 120. In the spray drier assembly 104, the plasma 112A is aerosolized and dried, producing a dried plasma that may be collected in the collection chamber 104C and stored for future use. Waste water 122 extracted from the plasma during the spray drying process is collected for removal from the system 100 (e.g., in a containment vessel 516).

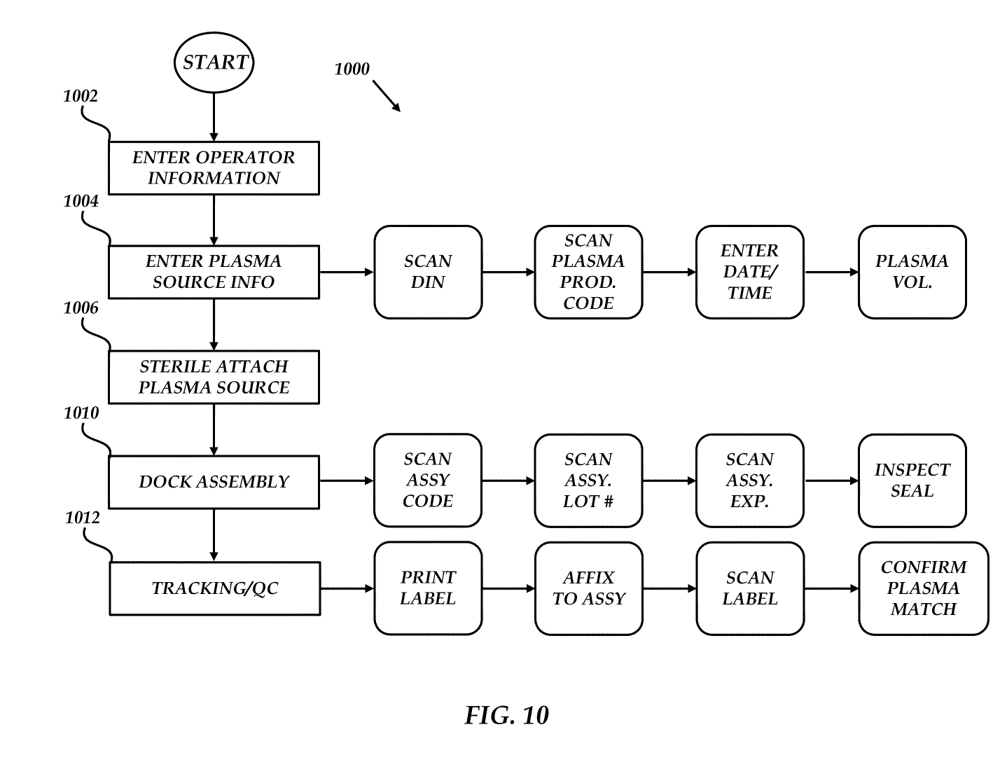

Embodiments of the spray drier device 102 may further include a spray drier spray drier computing device 124. As discussed in greater detail below with respect to FIGS. 10-12, the spray drier computing device 124 may include one or more computing devices configured to monitor and control a plurality of process parameters of the spray drying operation. The spray drier computing device 124 may be a local device integrated with the spray drier device 102 and/or a computing device in communication with the spray drier device 102 remotely.

The spray drier spray drier computing device 124 may include one or more user interfaces. For example, one user interface allows an operator to input data (e.g. operator information, sample information, spray drier assembly information, etc.), command functions (e.g., start, stop, etc.). Another example of a user interface may display status information regarding components of the spray drier device (e.g., operating normally, replace, etc) and/or spray drying process information (e.g., ready, in-process, completed, error, etc.). The spray drier device 102, in an aspect, includes spray drying computer 124 that allows the operator to perform the following operations: 1) to input relevant lot history information, 2) automate the spray drying process, 3) ensure dried product quality by evaluating real-time process parameters.

Embodiments of the spray drying computing device 124 may further communicate with a middleware controller 150 via a network to perform the following operations: 1) receive process and drying data from the spray drier, 2) match process data to donor plasma unit data, 3) store information in a database for record retention, and 4) transmit combined data to the blood center information system for record retention. Middleware controller 150 can operate with one or more spray drying computing devices 124.

The spray drier device 102 records one or more data associated with a spray drying operation. Examples of these data include, but are not limited to, bibliographic information regarding the liquid plasma which is spray dried (e.g., lot number, collection date, volume, etc.), bibliographic information regarding the spray drying operation (e.g., operator, date of spray drying, serial number of the spray drier assembly 104, volume of dried plasma, etc.), process parameters (e.g., flow rates, temperatures, etc.). Upon completion of a spray drying operation, the spray drier device 102 communicates with the remote computing system to transmit a selected portion or all the collected data to middleware control 150.

For example, spray drying system 100 can be housed in a blood bank facility. The blood bank facility receives regular blood donations for storage. Liquid plasma is separated from whole blood donations, dried using the spray drying system 100, and subsequently stored until use. Middleware controller 150 can be one or more computing devices maintained by the blood bank for tracking stored, dried blood. Providing a spray drying system 100 that relays information regarding dried plasma to local middleware controller 150 that is housed at the blood center allows such information to be conveyed and accessed quickly and accurately at the blood center.

In an alternative embodiment, illustrated in FIG. 1B, a plurality of spray drier systems 100A, 100B, . . . 100N can be used in combination with a pooled plasma source 112′. In general, the pooled plasma source 112′ is a bulk source of blood plasma having a volume larger than one blood unit, as known in the art (e.g., approximately 1 pint or 450 mL). Two or more of the spray drier systems 100A, 100B, . . . 100N can operate concurrently, each drawing blood for spray drying from the pooled plasma source 112′, rather than a smaller, local blood source (e.g., a single unit).

The spray drier systems 100A, 100B, . . . 100N in a pooled environment can operate under the control of a spray drier computing device 124′. The spray drier computing device 124′ is similar to spray drier computing device 124 discussed above, but configured for concurrent control of each of the spray drier systems 100A, 100B, . . . 100N. The spray drier computing device 124′ further communicates with a middleware computing device 150, as also discussed above.

In the pooled environment of FIG. 1B, the starting liquid plasma can be pooled to form the pooled source 112′ before drying. The pooled plasma source 112′ can treated for pathogen inactivation e.g., with UV light, a chemical, and the like. The flow of plasma 112A drawn from the pooled plasma source 112′ is dried using one or more spray drying systems 100 of the present technology and collected in a single collection chamber or a plurality of collection chambers. If the pooled plasma is dried for human transfusion, then each collection chamber can be configured with an attached rehydration solution. If the plasma dried from the pooled source 112′ is to be used for fractionation purposes, then it may be collected in a collection chamber configured without the rehydration solution.

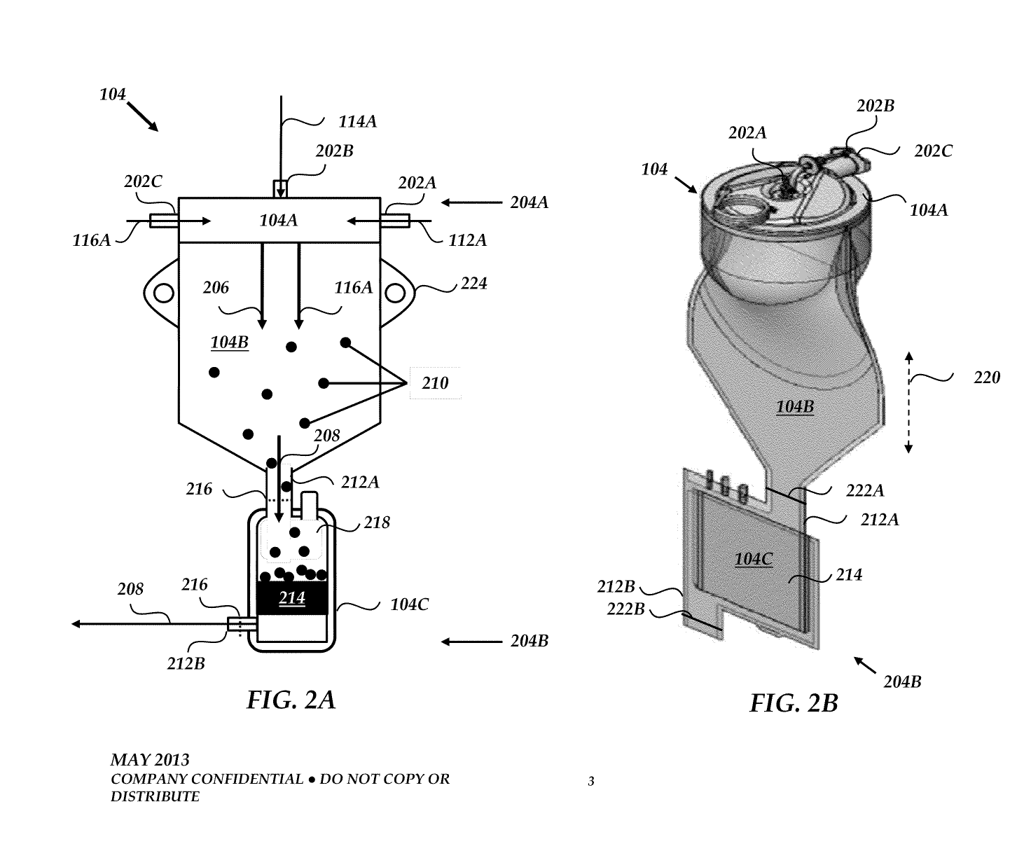

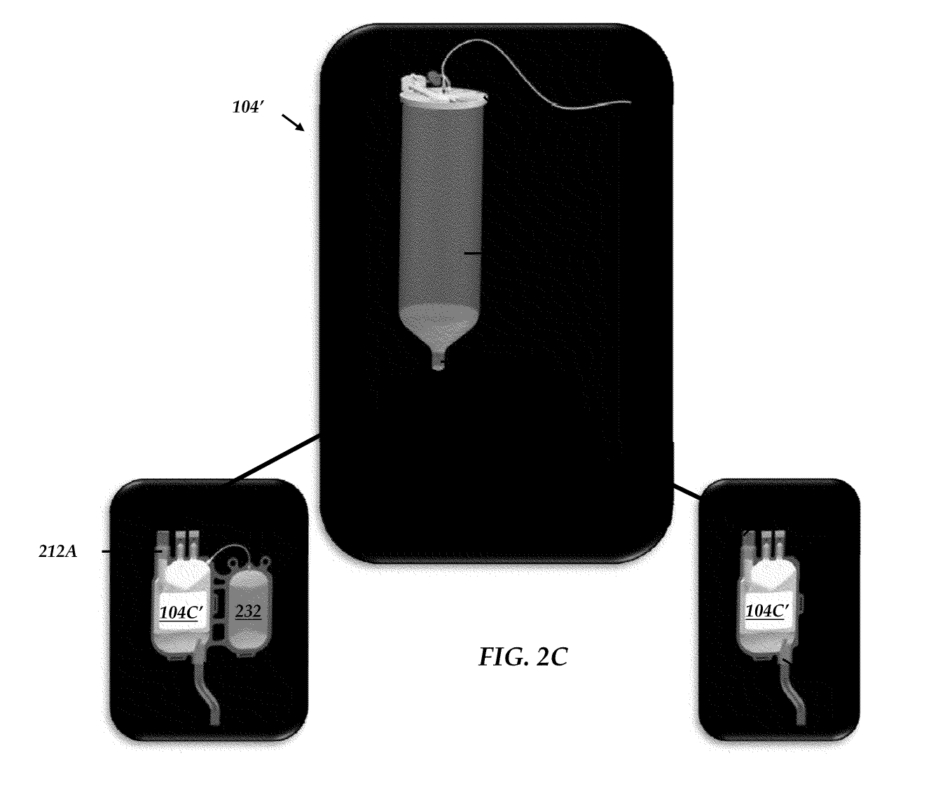

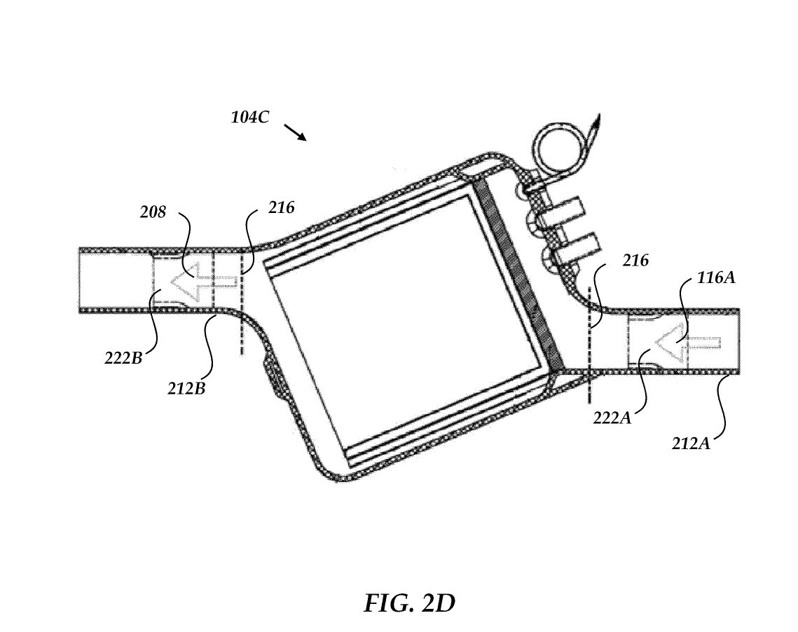

FIGS. 2A and 2B illustrate embodiments of the spray drier assembly 104 in greater detail, in schematic and three-dimensional, perspective views, respectively. The spray drier assembly 104 shown in FIGS. 2A and 2B includes a spray drying head 104A, a drying chamber 104B, and a collection chamber 104C in fluid communication. The spray drying head 104A shown is positioned adjacent to a first end 204A of the spray drier assembly 104. The collection chamber 104C is positioned at about the second end 204B of the spray drier assembly 104. The collection chamber 104B is positioned between the spray drier head 104A and the collection chamber 104C. In certain embodiments, the drying chamber 104B and collection chamber 104C are integrally formed (e.g., from the same material). In alternative embodiments, the drying chamber 104B and collection chamber 104C are separately formed (e.g., from different materials) and joined together.

The plasma is aerosolized to form aerosolized plasma 206 and emitted into the drying chamber 104B, where the flow of the aerosolizing gas 114A and the drying gas 116A direct the aerosolized plasma 206 towards the collection chamber 104C. In an embodiment, the aerosolized plasma 206 is dried to form dried plasma 210 in at least two stages. A first, initial drying stage occurs when the aerosolized plasma 206 is exposed to the flow of drying gas 116A in the drying chamber 104C. A second, subsequent drying stage occurs as the flow of aerosolized plasma 206 is directed into the collection chamber 104C, still in contact with the flow of drying gas 116A. It may be understood, however, that in alternative embodiments, a single drying stage may be employed, either the first or second drying stage.

In a further embodiment, if desired, the secondary drying can be performed in the collection chamber 104C by maintaining the drying gas flow 116A across the dried plasma 210 once it has been collected in the collection chamber 104C. In the case of secondary drying, some of the parameters for flow rates and temperatures of the drying gas can be changed from those specified for primary drying. For example, the flow of drying gas 116A can be heated to a temperature between about 35° C. and about 80° C., and can have a flow rate of between about 10 CFM to about 35 CFM. The flow of aerosolizing gas 114A can have a flow rate of between about 0 L/min and about 20 L/min and a temperature between about 15° C. to about 30° C. (e.g., 24° C.). In another embodiment, heat for the primary or secondary drying can be supplied by a heating device employing energy such as electromagnetic, radiofrequency, radiation, microwave waves that passes through the walls of the drying chamber 104B, the collection chamber 104C, or both.

In yet another embodiment, a desiccant can be placed within the collection chamber 104C to facilitate drying. For example, the desiccant or similar substance can be placed in contact with the dried sample. In another example, the desiccant or similar substance is not placed in contact with the dried plasma but rather in fluid communication with the dried plasma (e.g., on either side of the filter within the collection chamber 104C, in a separate pocket or port). Beneficially, use of desiccant within the collection chamber may allow for further moisture removal from the dried plasma over the duration of storage and increase the shelf-life of the dried plasma.

With further reference to FIGS. 2A and 2B, the spray drier head 104A includes a liquid sample inlet port 202A, an aerosolizing gas inlet port 202B, and a drying gas inlet port 202C for receiving respective flows of liquid sample 112A (e.g., blood plasma), aerosolizing gas 114A, and drying gas 116A. As discussed in greater detail below, in the spray drying head 104A, received flows of aerosolizing gas 114A and blood plasma 112A are mixed to form the aerosolized plasma 206. The aerosolized blood plasma 206 is further exposed to the drying gas 116A simultaneously upon aerosolization, as illustrated in FIG. 2A, or shortly thereafter (e.g., further in the drying chamber 104C and/or even in the collection chamber 104B).

In the drying chamber 104B, the aerosolized liquid plasma 206 and the flow of drying gas 116A remain in contact. Moisture is transferred from the aerosolized liquid plasma 206 to the drying gas 116A through evaporation. As the moisture transfers from the liquid plasma 206 into the flow of drying gas 208, humid gas 208 forms in the drying chamber 104C. The flow of the drying gas 116A directs not only the dried plasma 210 but also the humid gas 208 (e.g., air) to exit the spray drier assembly 104, as further described herein. In certain embodiments, the dried plasma 210 has a mean particle size particle size ranging between about 0.2 μm to about 25 μm. Beneficially, a dried plasma size of less than or equal to about 25 μm may provided improved rehydration performance over larger partials. In further embodiments, the drying chamber 104B can be in thermal communication with a heater 514′ (see, e.g., FIGS. 5A, 5B) configured to heat the flow of drying gas 116A to a selected temperature within the drying chamber 104B.

The humid air 208 and dried plasma 210 are further directed into the collection chamber 104C through an inlet port 212A connecting the collection chamber 104C and the drying chamber 104B. The collection chamber 104C includes filter 214 which allows through-passage of the humid gas 208 and inhibits through-passage of the dried plasma 210. The separation of humid gas 208 and the dried plasma 210 occurs when the humid gas 208 passes through filter 214, which retains the dried plasma 210 and allows the humid gas 208 to pass through the pores of the filter 214. The design of the collection chamber 104C allows the humid gas 208 to be exhausted from the collection chamber 104C through an exhaust port 212B, while the dried plasma 210 is retained in a reservoir 218 of the collection chamber 104C. In an embodiment, as the humid gas 208 and dried plasma 210 pass through the collection chamber 104C, the dried plasma 210 continues to lose moisture during the secondary drying stage.

Beneficially, by exhausting the humid air 208 from the collection chamber, through the filter 214 and exhaust port 212B provides a number of advantages. In one aspect, increased collection efficiency (i.e., less loss of dried plasma 210) may be achieved. In another aspect, the flow of humid air 208 through the collection chamber 104C may help in further removing moisture from dried plasma 210 already collected within the collection chamber 104C and increase the shelf-life of the dried plasma 210.

When desired, the operator can subsequently detach (e.g., cut) the collection chamber 104C from the spray drying assembly 104 and hermetically seal the collection chamber 104C at about the inlet and exhaust ports 212A, 212B (e.g., locations 216). This sealing process allows the collection chamber 104C to subsequently function as storage for the dried plasma 210 until use. Beneficially, by providing a collection chamber 104C for collecting dried plasma 210 that can be sealed and removed from the spray drying assembly 104, the need to further collect and remove the dried plasma 210 from the spray drier assembly 104 is eliminated to a containment and storage vessel. Furthermore, possible contamination of the dried plasma 210 in such a transfer process is avoided.

With reference to FIGS. 2B and 2D, the collection chamber 104C may additionally include a plurality of one-way valves 222A, 222B positioned at about the inlet port 212A and the exhaust port 212B, respectively. The one-way valve 222A may function to permit gas flow from the drying chamber 104B to the collection chamber 104C and inhibit gas flow from the collection chamber 104C to the drying chamber 104B. The one-way valve 222B may function to permit gas flow from the collection chamber 104C while inhibiting gas flow into the collection chamber 104C. While FIG. 2D shows the position of one-way valves 222A, 222B at both the inlet and exhaust ports 212A, 212B of the collection chamber 104C, it may be understood that a single one-way valve may be employed at either the inlet port 212A or the outlet port 212B of the collection chamber 104C.

Referring to FIG. 2C, in certain embodiments, an alternative spray drier assembly 104′ may be provided that is reusable. The spray drier assembly 104′ includes a reusable spray drier head 104A′, a reusable drying chamber 104B′, and a single-use (i.e., disposable) collection chamber 104C′. The drying chamber 104B′ has a distal end 230 to which to one or more disposable collection chambers 104C′ are connected. The disposable collection chambers 104C′ can be attached to the distal end 230 of the reusable drying chamber 104B′ using a removable attachment as known in the art which forms a hermetic seal between the reusable drying chamber 104B′ and disposable collection chamber 104C′. The collection chamber 104C′ can include an exhaust port 212B with one-way valve 222B to prevent the backflow of dried plasma 210. The reusable spray drier head 104′ and reusable spray drying chamber 104B′ may be independently formed from reusable materials including, but not limited to, metals, alloys, stainless steels, and the like.

In alternative embodiments, one or more of the spray drier head 104A, drying chamber 104B, and collection chamber 104C may be formed from disposable materials. Examples of disposable materials may include, but are not limited to, polymers.

As further illustrated in FIG. 2C, the collection chamber 104B′ may optionally have a rehydration solution carrier 232 attached. This embodiment lends itself to use with pooled plasma sources 112′ (e.g., FIG. 1B).

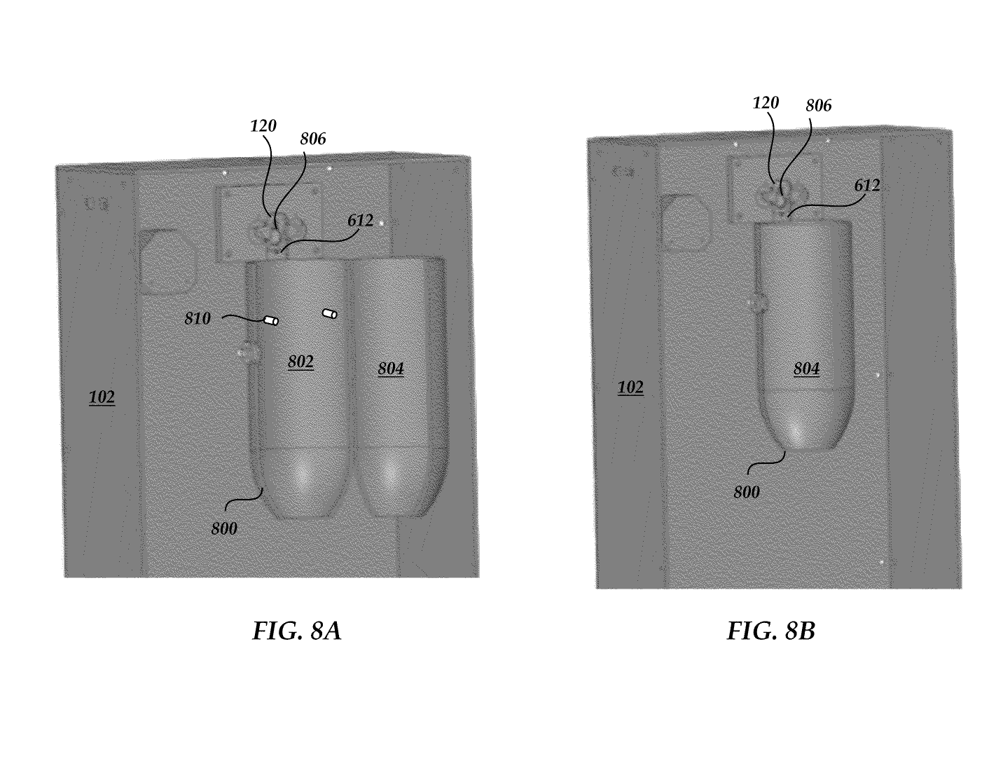

In additional embodiments, the spray drier assembly 104 may further include a plurality of guides 224 that provide a mechanism for placing the spray drier assembly 104 into a correct position to couple with the spray drier device 102. For example, in certain embodiments, the plurality of guides 224 may be positioned on the drying chamber 104B, the collection chamber 104C, or both. The guides 224 may be adapted to mate with corresponding guides positioned on the spray drier device 102 for alignment of the spray drier assembly 104, as discussed in greater detail below with respect to FIG. 8. While the guides 224 are illustrated in FIG. 2A as being positioned on the drying chamber 104B, it may be understood that the guides 224 may be positioned anywhere upon the spray drier assembly 104, as necessary.

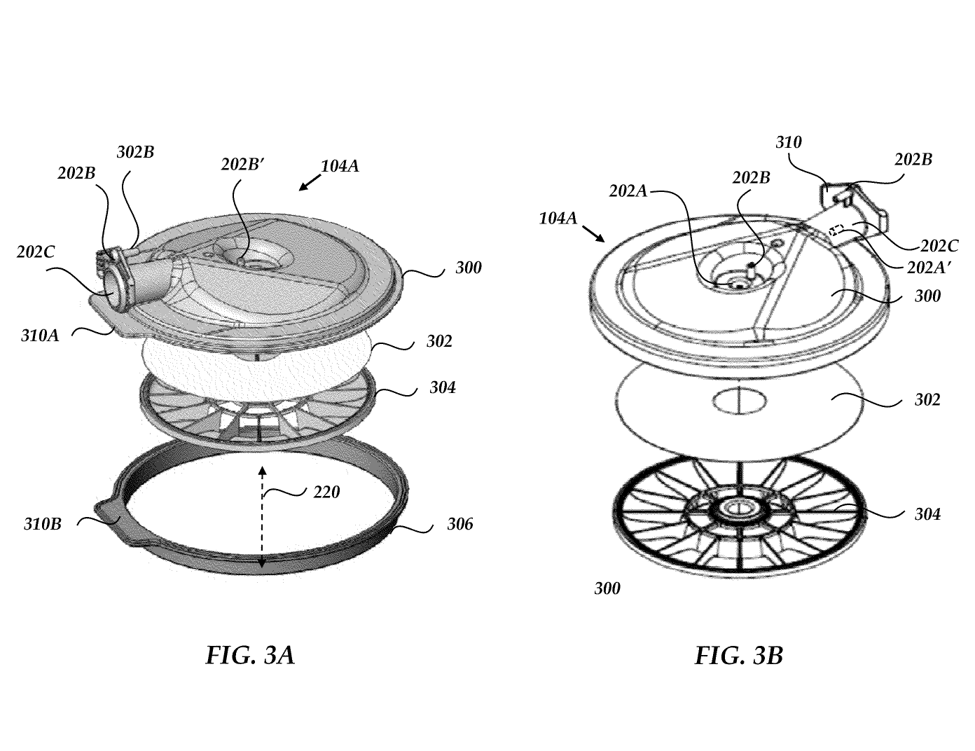

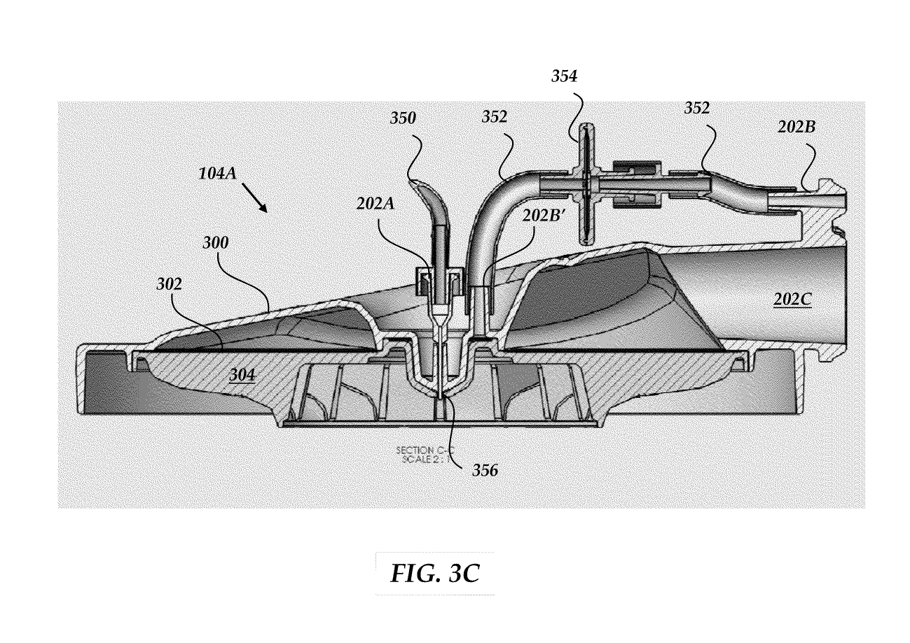

Embodiments of the spray drying head 104A are illustrated in additional detail in FIGS. 3A-3C. FIGS. 3A, 3B illustrate a spray drying head design, in front and rear view, which include a spray head 300, a filter 302, and a filter support 304. The filter 302 may be interposed between the spray head 300 and the filter support 304 for filtering the flow of drying gas 116A. In certain embodiments, the filter 302 may be a 0.2 μm filter. In certain embodiments, the filter 302 may be omitted. For example, in circumstances where the flow of drying gas 116A is determined to be sufficiently clean/sterile for use without filtering by filter 302.

The spray head 300 and filter support 304 may be connected together, for example, by welding, to form a head/filter sub-assembly. An adaptor 306 may be further provided and connected (e.g., welded) to an integrally formed drying chamber 104B and collection chamber 104C at the end of the drying chamber 104B opposite the collection chamber 104C to form a chamber/adaptor sub-assembly. The head/filter sub-assembly and the chamber/adaptor sub-assembly may be connected to each other to form the spray drying assembly 104. In further embodiments, each of the spray head 300 and adaptor 306 may include respective flanges 310A, 310B to facilitate connection of the head/filter sub-assembly and the chamber/adaptor sub-assembly.

FIG. 3C shows a cut-away view of the spray drier head 300 for illustration of different flow pathways within the head 300. A plasma conduit 350 may be provided which places the plasma inlet 202A in fluid communication with the plasma source 112. In certain embodiments, where the flow of plasma 112A does not pass through the dock 120, the plasma conduit 350 may provide a direct connection between the plasma inlet 202A and the plasma source 112. In other embodiments, where the flow of plasma 112A does pass through the dock 120, the plasma conduit 350 may provide a fluid connection between the plasma inlet 202A and the plasma source via a second plasma inlet 202A′ (see, e.g., FIG. 2B) and the dock 120.

An aerosol gas conduit 352 may be further provided places the aerosolizing gas inlet 202B in fluid communication with a second aerosolizing gas inlet 202B′ located at about the center of the head 300. In certain embodiments, a filter 354 may also be placed inline with the conduit 352. The filter 354 may be selected, as appropriate, for the desired degree of filtering. For example, the filter 354 may be a 0.2 μm filter.

The flow of plasma 112A provided via conduit 350 and the flow of aerosolizing gas 114A provided via conduit 352 meet at a nozzle 356 of the spray drier head 300 to produce the aerosolized plasma 206, which is subsequently ejected from the head 300. This aerosolized plasma 206 is further brought into contact with the flow of drying air 116A received at the drying gas inlet 202C and exiting the spray drier head 300.

In alternative embodiments, the flow of aerosolizing gas 114A may be omitted from the system 100. Instead, the nozzle 356 may include an aerosolizer in communication with the plasma inlet 202A and receive the flow of plasma 112A. In one example, the aerosolizer may be adapted to aerosolize the flow of plasma 112A using ultrasonic waves at a selected wavelength and frequency. In another example, the aerosolizer may be an ultrasonic atomizing transducer, an ultrasonic humidified transducer, or a piezo ultrasonic atomizer.

Use of the aerosolizer may provide a number of benefits. In one aspect, use of the aerosolizer may eliminate the need for the flow of aerosolizing gas 114A and filter 354, simplifying the spray drier assembly 104 and reducing its cost. In another aspect, eliminating the flow of aerosolizing gas 114A may remove a possible contamination source from communication with the spray dryer assembly 104.

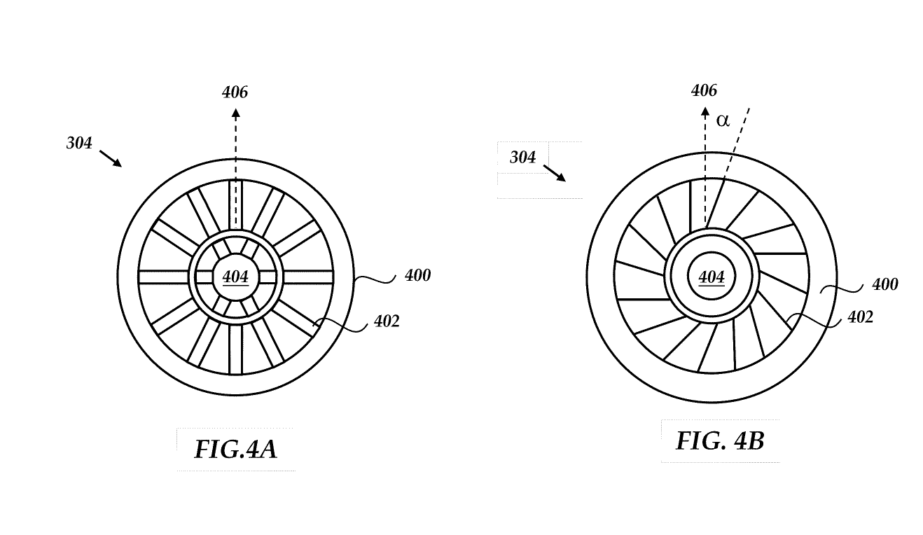

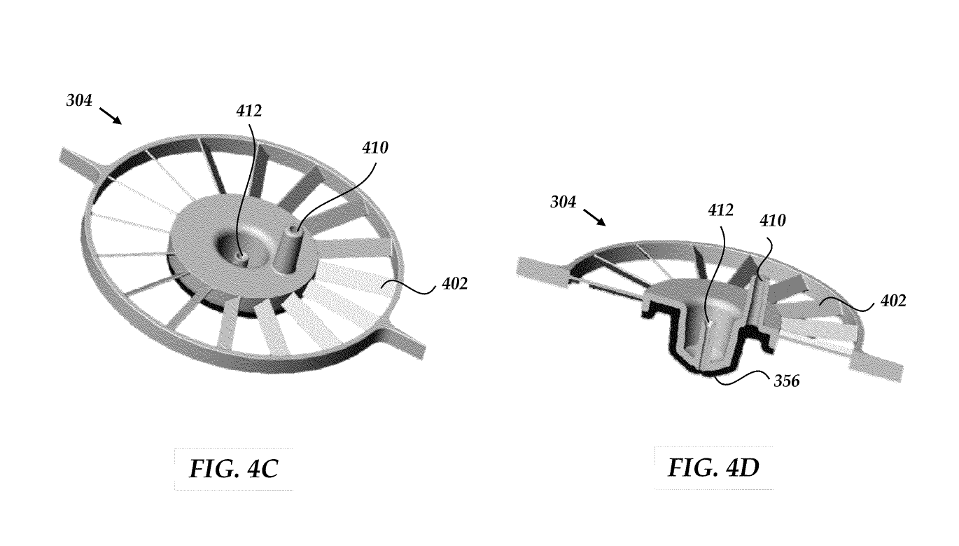

FIGS. 4A and 4B illustrate embodiments of the filter support 304 in a top down view. The filter support 304 includes a frame 400 having a shape configured for attachment with the spray head 300 and a plurality of fins 402. For example, the frame 400 may be formed in a circular configuration for attachment with a circular spray head 300. However, it may be understood that the spray drier head 300 and frame 400 may adopt other shapes, as desired. The fins 402 may also extend outward from a frame center 404 at a selected angle α with respect to a surface normal 406 from the frame center 404. For example, FIG. 4A illustrates one embodiment where angle α is approximately zero and the fins 402 extend radially outward from the frame center 404. FIG. 4B illustrates another embodiment where the angle α is non-zero and the fins are angled circumferentially about the frame center 404. In certain embodiments, angle α may range between about 20 degrees to about 60 degrees, preferably about 45 degrees. In other embodiments, the fins 402 may be set at angular orientations where a surface normal to the plane of the fins 402 lies parallel to the plane of the filter 302, perpendicular to the plane of the filter 302, and angular orientations there between. For example, FIGS. 4C, 4D, illustrate fins 402 oriented at an angle with respect to the plane of the filter 302.

By adjusting the orientation of the fins 302, the filter support 304 may modify the laminar flow of the aerosolized plasma 206 and drying gas 116A passing there through to create a helical flow path. The helical flow path may possess any number of rotations. For example, the aerosolized plasma and drying gas 116A may be directed into a helical swirl having a selected number of revolutions through the length of the drying chamber 104B (e.g., ¼ revolution, 1 revolution, 5 revolutions, 15 revolutions, 25 revolutions, etc.).

With further reference to FIGS. 4C, 4D, additional embodiments of the filter support 304 are illustrated, in perspective and cut-away views, respectively. The filter support 304 may further include a first channel 410 that in fluid communication with the aerosolizing gas inlet port 202B of the spray head 300 and a second channel 412 in fluid communication with the liquid sample inlet port 202A of the spray head 300. The first and second channels 410, 412 direct the flow of blood plasma 212A flow of aerosolizing gas 214A to a nozzle 412, where the flows are mixed to form the aerosolized blood plasma 206 and emitted into the drying chamber 140B.

Producing a helical flow path for the drying gas 116A is believed to provide benefits to the spray drying process. For example, the helical flow path may increase contact of the spray drying gas 116A with the aerosolized liquid sample 206 (e.g., aerosolized blood plasma). This increased time of contact may reduce the path length travelled by the aerosolized liquid sample 206 to achieve a given level of dryness, allowing the length of the drying chamber to be reduced. The increased time of contact may also reduce the time required to achieve a given level of dryness.

With continued reference to FIGS. 3A, 3B, the spray head 300 may include the plasma inlet port 202A, the aerosolizing gas inlet port 202B, and the drying gas inlet port 202C. In certain embodiments, the spray head 300 may further include aerosolizing gas inlet port 202B′ which is in fluid communication with the aerosolizing gas inlet port 202B via conduit 352.

In certain embodiments, the plasma inlet port 202 may be in direct fluid communication with the liquid sample source 112. In this case, the flow of liquid sample 112 does not travel through the dock 120. In additional embodiments, the spray head 300 may also include the plasma inlet port 202A′, which is in fluid communication with the plasma inlet port 202. In this case, the flow does flow through the dock 120.

In certain embodiments, one or more of the liquid sample inlet port 202A, the aerosolizing gas inlet port 202B, and the drying gas inlet port 202C may not be co-axial with respect to each other. The use of non-co-axial ports may, beneficially, reduce the likelihood of leak paths between the respective sources of liquid sample, 112, aerosolizing gas 114 and drying gas 116, as a leak in one flow path is inhibited from flowing into another flow path. Furthermore, so configured, a leak in one flow path is easier to detect, since the flow paths are isolated from one another.

The spray head 300 may further include a plurality of flanges 310 positioned at about the periphery of the drying gas inlet port 202C. As discussed in greater detail below with respect to FIGS. 6A-6B, 7A-7B, the plurality of flanges 310 may be engaged by a locking mechanism when the spray drier head is positioned within the dock 120. With the locking mechanism so engaged, the spray drier assembly 104 may be inhibited from removal from the dock 120.

In an embodiment, the spray drying head 104A and the drying chamber 104B are designed to be used for numerous spray drying operations, sterilized (e.g., autoclaved) prior to each run. For example, in this case, the spray drying head 104A and drying chamber 104B be formed from reusable, re-sterilizable materials, including, but not limited to, metals and alloys (e.g., stainless steel, titanium, aluminum, silver, and the like). Additionally, re-sterilizable material can be made from polymeric materials such as silicon, rubber and plastic. In alternative embodiments, the spray drying head 104A and the drying chamber 104B are designed to be sterilized (e.g., irradiation, or autoclaved) and used in a single spray drying operation. For example, in this case, the spray drying head 104A and drying chamber 104B may be formed from disposable, sterile materials, including, but not limited to, polymers, stainless steel, or silicon.

The discussion will now turn to FIG. 5A, which illustrates an embodiment of the spray drier device 102 in combination with the spray drier assembly 104, detailing flow of the blood plasma 112A, aerosolizing gas 114A, and the drying gas 116A through the spray drier device 102 and assembly 104 to produce dried plasma 210.

The flow of plasma 112A may be routed to the spray drier assembly 104 in the following manner. In an embodiment, the flow of blood plasma 112A originates from the blood plasma source 112. In certain embodiments, the source of blood plasma 112 may be a single unit source (e.g., approximately 1 pint or 450 mL). However, in alternative embodiments, as discussed above with respect to FIG. 1B, the plasma source may be a pooled source 112′. The source of blood plasma 112 is brought into fluid communication with the spray drier device 102 at a sterile connection 502. A pump 504 may urge the flow of blood plasma 112A through the spray drier device 102 and to the spray drier assembly 102 at a selected rate according to the spray drier computing device 124.

From the sterile connection 502, the flow of plasma 112A may be provided directly to the spray drier assembly 104 or via the dock 120. In the former case, the spray drier device 102 may include a conduit extending from the sterile dock 502 to liquid sample inlet port 202A of the spray drier assembly 104. In the latter case, the flow of plasma 112 further passes through the plasma inlet 506A of the dock 120 to the plasma inlet port 202A′ of the spray drier assembly 104. From the inlet port 202A′, the flow of plasma 112 further travels to the inlet port 202A of the spray drier assembly 104 (e.g., via conduit 350). In certain embodiments, a filter may be interposed between the inlet port 202A′ and the inlet port 202A.

The flow of aerosolizing gas 114A is routed to the spray drier assembly 104, via the dock 120 of the spray drier device 102, in the following manner. The flow of aerosolizing gas 114A originates from the aerosolizing gas source 114. The aerosolizing gas may include, but is not limited to, compressed air, or an inert gas (e.g., nitrogen, carbon dioxide). The source of aerosolizing gas 114 is brought into fluid communication with the spray drier device 102 at an aerosolizing gas inlet 506B of the dock 120. The flow of aerosolizing gas 114A may be subsequently routed from the dock 120 to the aerosolizing gas inlet port 202B of the spray drier assembly 104.

The flow of drying gas 116A is routed to the spray drier assembly 104, via the dock 120 of the spray drier device 102, in the following manner. The flow of drying gas 116A originates from the drying gas source 116. The drying gas source 116 may be ambient environment (e.g., air). Further examples of drying gas source 116 include compressed air, and inert gases (e.g., nitrogen, carbon dioxide, etc.). A drying gas intake 508 may receive the flow of drying gas 116A from the drying gas source 116. The flow of drying gas 116A may be further routed to a drying gas inlet 506C of the dock 120 via one or more drying gas conduits. The flow of drying gas 116A may be subsequently routed from the dock 120 to the drying gas inlet port 202C of the spray drier assembly 104. A pump 504B may be used to urge the flow of drying gas 116A from the drying gas source 116 to the drying gas inlet 506C of the dock 120 at a selected rate according to the spray drier computing device 124.

In certain embodiments, a plurality of conditioners may be interposed between the drying gas source 116 and the dock 120. The conditioners may be configured to adjust one or more of the physical parameters of the drying gas, including, but not limited to, temperature, flow rate, and humidity. For example, the plurality of conditioners may include one or more of pump 504B, a heater 514 configured to heat the flow of drying gas 116A to a selected temperature, and a humidifier/dehumidifier 512 configured to add or remove water from the flow of drying gas 116A to achieve a desired humidity therein. Types of dehumidifiers include cold plate dehumidifiers, membrane dehumidifiers, mechanical separation dehumidifiers and the like. Water extracted from dehumidification may be removed from the spray drier device 102 to containment vessel 516 for disposal.

The discussion will now be directed to embodiments of the disclosure that provide the system 100 with a functionally closed environment for inhibiting contaminants from entering the spray drier device 102 and spray drier assembly 104. With respect to the flow of plasma 112A, the sterile connection 502 maintains the closed environment between the plasma source 112 and the dock 120. With respect to the flow of the aerosolizing gas 114A, a plurality of filters 510B may be interposed between the aerosolizing gas source 114 and the dock 120 to maintain the closed environment between the aerosolizing gas source 114 and the dock 120.

With respect to the drying gas 116A, a plurality of filters 510C and 520 may be may be interposed between the drying gas source 116 and the dock 120 to maintain the closed environment between the drying gas source 116 and the dock 120. In certain embodiments, the plurality of filters 510B, 510C can be configured to provide a combined BFE of equal or greater than 10⁶. In alternative embodiments, a separate filter can also be employed between the aerosolizing gas source 114 and the dock 120. Beneficially, the plurality of filters 510C, 520 may provide that the flow of drying gas 116A received at the dock 120 is sufficiently cleaned from its initial state in the environment (e.g., drying gas source 116) to produce transfusion grade dried plasma. Furthermore, the other conditioners (e.g., humidifier/dehumidifier 512, pump 504B, heater 514) may allow the spray drier device 102 to pre-treat the filtered air, isolating the spray drier device 102 from environmental conditions, and allowing the spray drier device 102 to operate in a variety of environments.

The manner in which the spray drier system 100 maintains the closed environment between the dock 120 and the spray drier assembly 102 will now be discussed. In one aspect, the spray drier assembly 104 may be provided for use with the spray drier device 102 in a sterile state. In another aspect, the dock 120 may be aseptically cleaned prior to receiving the spray drier assembly 104. In a further aspect, the spray drier assembly 104 and dock 120 may be directly connected, with no intermediate conduits.

While the connection between the spray drier assembly 104 and the dock 120 is an aseptic connection, the spray drier device 102 further includes environmental controls to reduce the likelihood of environmental or bacterial contamination between the spray drier assembly 104 and the spray drier device 102 at the dock 120. For example, environmental controls can be provided to produce an environment at and around the spray drier device 104 and dock 120 with a bacterial load of about 200 CFU/m³ or less. With continued reference to FIG. 5A, the environmental controls may include a fan that provides a unidirectional airflow 530, directed through an environmental chamber filter 532, towards the connection between the spray drier assembly 104 and the dock 120. The environmental chamber filter 532 may possesses a high efficiency particulate air (HEPA) efficiency of at least 99.99%. In this manner, a clean environment is provided about the connection between the spray drier assembly 10⁴ and the dock 120. Furthermore, the unidirectional airflow 530 produces a positive pressure in areas outside of the connection between the spray drier assembly 104 and the dock 120 that inhibits contaminants from entering areas at or adjacent to this connection. Furthermore, by increasing the HEPA filtration efficiency, the environmental load around the connection between the spray drier assembly 104 and the dock 120 can be further reduced.

In order to further reduce the likelihood of contamination at the connection between the spray drier assembly 104 and the dock 120, additional processes may be performed. In one process, the flow rates of the aerosolizing gas 114A and the drying gas 116A may be varied between idle and operating states of the system 100. For example, when the system 100 is idle (e.g., the flow of plasma 112A is not provided to the spray drier assembly 104), the flow of aerosolizing gas 114A and the drying gas 116A may provided at a reduced flow rate as compared to when the system 100 is operating (e.g., the flow of plasma 112A is provided to the spray drier assembly 104). In this manner, the collection of bacteria in the gas lines and around the spray drier assembly 104 may be further minimized. In another process, leak testing of the connection between the spray drier assembly 104 and the dock 120, as well as the spray drier assembly 104 itself, may be performed prior to the start of spray drying operations to ensure no leaks are present.

As spray drying operations are being performed, the flow of drying gas 116A moves through the spray drying assembly 104, initially as drying gas 116A, then later as humid drying gas 208, as moisture is transferred from the flow of plasma 112A to the drying gas 116A. As illustrated in FIG. 5A, the humid drying gas 208 is separated from the dried plasma 210 in the collection chamber 104C by the filter 214 and exits the collection chamber 104 through the exhaust port 212B. The humid drying gas 208 may be passed through a filter 540 and a plurality of conditioners 544 which return the humid drying air 208 to a state having reduced humidity and contaminants. For example, contaminants may enter the humid drying air 208 in the event that the spray drier assembly 104 is compromised. In this manner, the humid drying air 208 is of a quality suitable for venting to environment 546. In an embodiment, the plurality of conditioners 544 includes a dehumidifier which transmits waste water to the containment vessel 516. Accordingly, the humidity of the drying air 208 may be reduced such that moisture in this exhausted air does not over-saturate the environment 546 surrounding the spray drier device 102.

The discussion will now turn to venting and sealing of the collection chamber 104C. Upon completion of the spray drying process, a significant amount of humid drying gas 208 remains within the collection chamber 104C. If the majority of the humid drying gas 208 is not removed from the collection chamber 104C prior to sealing, the collection chamber 104C occupies a relatively large volume, compared to a deflated state, with an increased likelihood of rupture due to internal pressure or puncture. Accordingly, it is desirable to purge the humid drying gas 208 from the collection chamber 104C prior to sealing using a purging mechanism.

In one embodiment, the purging mechanism may be a pump 542, configured to operate as a vacuum pump. For example, the collection chamber 104C may be initially sealed at about the inlet port 212A. The humid drying gas 208 within the collection chamber 104C may be expelled to atmosphere 546 by the vacuum generated by the pump 542. Owing to the one-way valve 222B positioned within the exhaust port 212B, the humid drying gas 208 is inhibited from re-entering the collection chamber 104C via the exhaust port 212B once expelled. After removing the desired amount of humid drying gas 208 from the collection chamber 104C, the collection chamber 104C may be sealed at about the exhaust port 212B.

In another embodiment, the purging mechanism may include at least two plates 550 configured to compress the collection chamber 104C. The at least two plates 550 may be disposed at opposing sides of the collection chamber 104C and configured to move between a first position and a second position. In the first position, the at least two plates 550 do not exert a compressive force upon the collection chamber 104C. In the second position, the at least two plates 550 are moved towards one another so as to exert a compressive force upon the collection chamber 104C that urges at least a portion of the humid drying air 208 from the collection chamber 104C.

When employing the plurality of plates 550 to express humid drying gas 208 from the collection chamber 104C, the collection chamber 104C may be initially sealed at about the exhaust port 212B. Subsequently, the humid drying gas 208 within the collection chamber 104C may be expelled to the drying chamber 104B by the mechanical force of the plurality of plates 550. Owing to the one-way valve 222A positioned within the inlet port 212B, the humid drying gas 208 is inhibited from re-entering the collection chamber 104C via the inlet port 212A once expelled. After removing the desired amount of humid drying gas 208 from the collection chamber 104C, the collection chamber may be sealed at about the inlet port 212A.

FIG. 5B illustrates an alternative embodiment of the system 100′ in which humid drying gas 208 expelled from the collection chamber 104C is filtered, conditioned, and recycled for use as a drying gas source 116′. For example, the humid drying gas 208, after being expelled from the collection chamber 104C, may be passed through the filter 540 and the plurality of conditioners 544 as discussed above with respect to FIG. 5A. The reconditioned drying gas exiting the plurality of conditioners 544 is thereby restored to a sterile, less humid state suitable for further use as drying gas source 116′. This drying gas source 116′ is provided in fluid communication with pump 504B. In other respects, the system 100′ operates in the manner discussed above with respect to system 100.

The systems 100, 100′ of FIGS. 5A, 5B each have respective advantages. For example, with respect to system 100, in the event filter 304 fails, the likelihood of cross-contamination between the spray drier assembly 104 and the flow of drying gas 116A/humid drying air 208 may be reduced by exhausting the humid drying air 208 from the system 100. In another example, with respect to system 100′, the flow of drying gas 116A is isolated from the environment surrounding the spray drier system 100′, simplifying the complexity of the system 100′ Furthermore, conditioning of the flow of drying gas 116A (e.g., temperature, flow rate, humidity, etc.) may be reduced or eliminated, improving the efficiency of system 100′.

The discussion will now turn to embodiments of the dock 120 and coupling of the dock 120 with the spray drier assembly 104. FIGS. 6A, 6B illustrate front and rear views of the dock 120, respectively. The front of the dock 120 is configured to receive the spray head 300 of the spray drier assembly 104 and may include a backplate 602 upon which are mounted a plurality of input ports 506B, and 506C. The input port 506B may be configured to receive the aerosolizing gas inlet port 202B for fluid communication between the aerosolizing gas source 114 and the dock 120. The input port 506B may also be configured to receive the drying gas inlet port 202C for fluid communication between the drying gas source 116 and the dock 120.

The input ports 604B, 604C may further extend through the dock 120 from the front side to the rear side. On the rear of the dock 120, the input ports 604B, 604C are configured to mate with the inlet ports 202B, 202C, respectively, to receive the flows of aerosolizing gas 114A and drying gas 116A.

A locking mechanism 610 in communication with an actuator 612 may be further positioned adjacent to the ports 604B, 604C. In general, the locking mechanism 610 may be reversibly moved between a disengaged position, where the locking mechanism 610 allows the spray drier assembly 104 to be freely added or removed from the dock 120, and an engaged position, where the locking mechanism 610 inhibits the spray drier assembly 104 from being removed from the dock 120.

For example, the locking mechanism 610 may include a plurality of cams in communication with a first plurality of pulleys 614 mounted on rods 616. The actuator 612 may include linear actuator (e.g., a piston) having a first end 620A and a second end 620B. The first end 620A of the actuator 612 may include a clevis rod end 622 in communication with a second pulley 624. The second end 620B of the actuator 612 may be in communication with a mechanism (e.g., a foot pedal or button, 110) which urges the clevis rod end 622 to extend (e.g., movement upwards) or retract (e.g., movement downward) when depressed and released, respectively.

When the mechanism 110 is depressed, the linear actuator may move upwards, causing the clevis rod end 622 to rotate the second pulley 624 in a first direction. The second pulley 624 may be in further communication with the first plurality of pulleys 614, where rotation of the second pulley 624 in the first direction may cause the plurality of cams to rotate away from the input ports 506B, 506C and adopt the disengaged position (see, e.g., FIG. 7A). In this disengaged position, the aerosolizing gas inlet port 202B′ and drying gas inlet port 202C of the spray drier assembly 104 are in fluid communication with the aerosolizing gas port 506B and the drying gas port 506C of the dock 120. The flanges 310 of the spray head 300 may be further positioned adjacent to the disengaged cams.

When the mechanism 110 is released, the linear actuator may move downwards, causing the clevis rod end 622 to rotate the second pulley 624 in a second direction, opposite the first direction. Rotation of the second pulley 624 in the second direction may cause the plurality of cams to rotate towards the input ports 506B, 506C and adopt the engaged position (see, e.g., FIG. 7B). In this engaged position, the aerosolizing gas inlet port 202B′ and drying gas inlet port 202C of the spray drier assembly 104 remain received by the aerosolizing gas port 506B and the drying gas port 506C of the dock 120. The flanges 310 of the spray head 300 are further covered by the cams in the engaged position. As a result, the spray head 300, and therefore the spray drier assembly 104, is inhibited from being removed from the dock 120 when the locking mechanism 610 is in the engaged position.

The discussion will now turn to FIGS. 8A, 8B, which illustrate embodiments of a cover 800 for the spray drying assembly 104. The cover 800 may be configured to contain at least a portion of the drying chamber 104B, collection chamber 104C, or both.

The cover 800 may include a first cover member 802 and a second cover member 804 coupled to one another. For example, the first and second cover members 802, 804 is formed in a clamshell design configured to move between an open position and a closed position with a hinge. As shown in FIG. 8A, cover 800 is mounted to the frame to the spray drier device 102. In the open position, the cover 800 is configured to receive the drying chamber 104B and attached collection chamber 104C of the spray drier assembly 104. The cover 800 can be adapted for mechanical removal and replacement so as to limit contamination of the spray drying assembly 104 and/or spray drying dock 120.

The cover 800 may be further dimensioned to contain the drying chamber 104B and collection chamber 104C in the closed position. For example, the cover may contain a volume smaller than the maximum inflation volume of the drying chamber 104B and collection chamber 104C. So dimensioned, when the drying chamber 104B and collection chamber 104C are inflated under pressure of the flow of drying gas 116A during spray drying operations, the exterior surface of the drying chamber 104B and collection chamber 104C may contact the inner surfaces of the cover 800. As a result, the cover 800 provides support to the drying chamber 104B and collection chamber 104C when inflated under internal pressure and inhibits undesired deformation and/or rupture of the drying chamber 104B or collection chamber 104C.

Beneficially, the use of the cover 800 as an external support for the drying chamber 104B and/or the collection chamber 104B may reduce the likelihood of rupture of these components. For example, with the cover 800 in place, deformation of the drying chamber 104B and/or collection chamber 104C due to internal pressure, creep (i.e., time dependent deformation at elevated temperature under load, such as the internal pressure), and the like, which may lead to rupture, can be inhibited.

Also, by allowing the drying chamber 104B and/or the drying chamber 104C to be inflated, under internal pressure, to conform to the shape of the cover may provide further benefits. For example, creases or pleats, which might trap plasma or otherwise alter the drying process, may be reduced or eliminated.

Furthermore, this configuration may also allow safe use of drying chambers 104B and/or collection chambers 104C fabricated from materials that are thinner than would otherwise be prudent. That is to say, the support provided by the cover 800 allows thinner (i.e., weaker) materials to be safely employed in fabrication of the drying chamber 104B and/or collection chamber 104C. Cost savings may also be realized by use of a thinner materials in the components of the spray drier assembly 104, lowering the cost of the assembly 104.

The cover 800 may further include a plurality of guides 810. The guides 810 may be adapted to mate with the plurality of guides 224 of the spray drier assembly 104 for proper alignment of the spray drier assembly 104 with the spray drier device 102 (e.g., positioning of the drying chamber 104C and the spray drying head 104A). For example, the plurality of guides 224 may be configured as apertures and the plurality of guides 810 may be configured as posts. Beneficially, by providing correct alignment of the spray drier assembly 104 with the spray drier device 102, guides 224 and 810 may reduce the likelihood of misalignment, which can lead to spray of the flow of plasma 112 on the walls of the drying chamber 104C

The dock 120 may further include a plurality port covers 806 (see FIGS. 8A, 8B) for covering the aerosolizing gas port 506B and the dryer gas port 506C when the spray drier assembly 104 is not positioned in the dock 120. Optionally, the plurality of port covers 806 may further cover the plasma port 506A of the dock 120, when present. In certain embodiments, the plurality of port covers 806 can be pierceable, frangible, or mechanically removed by the dryer dock so as to limit contamination of the spray drying assembly 104 and/or 120.

One or more sensors 612 may be further provided in communication with the plurality of port covers 806 and in communication with the actuator. The sensors 612 may be adapted to detect the presence or absence of the plurality of port covers 806. The sensors 612 can be mechanical, optical, magnetic, or electrical. Examples include, but are not limited to, optical sensors coupled with software/electrical disconnects, mechanical interlocks, limit switches, and the like.

In operation of the spray drier system 100, the plurality of sensors 612 may detect if any of the plurality of port covers 806 has been removed or compromised when the locking mechanism 610 is disengaged (e.g., when the spray drier assembly 104 is not present in the dock 120). Should the plurality of sensors 612 detect that one or more of the port covers 806 has been removed or compromised when the locking mechanism 610 is disengaged, the spray drier device 102 may not allow the spray drying process to be performed. In one example, the locking mechanism 610 may not be allowed to engage when the spray drier assembly 104 is placed in the dock 120. In another example, the computing device 124 may not allow an operator of the system 100 to enter a command to start spray drying operations. Alternatively, should the plurality of sensors 612 detect that the plurality of port covers 806 have remained in place and uncompromised when the locking mechanism 610 is disengaged, the spray drier device 102 may signal the operator that the spray drier assembly 102 may be coupled with the dock 120 and the plurality of port covers 806 may be removed. In certain embodiments, removal/puncture of the plurality of port covers 806 may be performed as an automated procedure by the spray drier device 102.

Beneficially, in this manner, the conduits conveying flows of the aerosolizing gas 114A, drying gas 116A, and, optionally, the plasma 112A from their respective sources may be kept free of contamination via the input ports 506A, 506B, 506C on the dock. This, in turn, may inhibit contaminants from entering the spray drier assembly 104.

In an alternate embodiment, the mechanism 110 can be replaced with a second sensor for sensing that determines proximity and/or engagement of the flange 310 of the spray drier assembly 104. As described above, the second sensor can also be mechanical, optical, magnetic, or electrical. The connection between the spray drier assembly 104 and the dock 120 is otherwise as described herein.

The cover 800 can further include alignment mechanism to allow proper positioning of the spray drier assembly within the spray drier assembly cover. In an embodiment, the alignment mechanism is mechanical. Examples of such alignment mechanisms can include complimentary slots and tabs, pins and bosses, and the like.

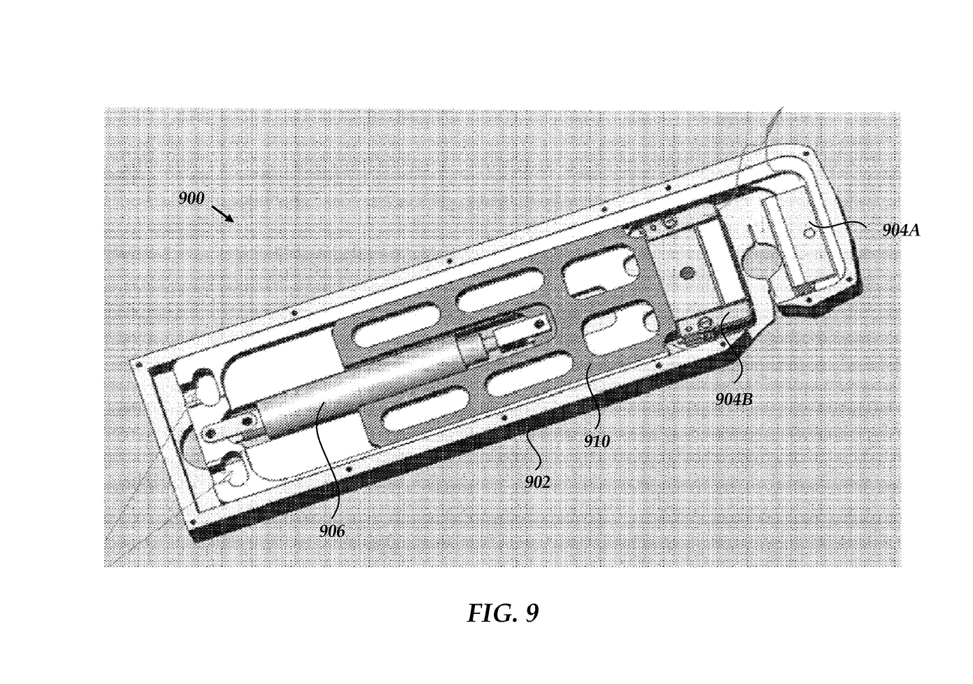

The spray drier device 102 also includes a plurality of sealing mechanisms 900 for sealing the collection chamber 104C after spray drying operations are complete. One embodiment of a sealing mechanism 900 is illustrated in FIG. 9. The sealing mechanism 900 may include a frame 902 and a pair of jaws 904A, 904B.

The upper and lower jaws 904A and 904B may be moveable with respect to the frame 900 and in concert with respect each other. They move a similar distance in opposite directions in a coupled motion such that when activated they advance toward one another to make the seal and when deactivated they retract apart. For example, a linear actuator 906 is in communication with a slide arm 910 abutting the lower jaw 904B. The linear actuator 906 may urge the slide arm 910 towards the upper jaw 904A, which in turn urges the lower jaw 904B towards the upper jaw 904A (e.g., into an engaged position). The linear actuator 906 may also move the opposite direction, urging the slide arm 910 away from the upper jaw 904A, which in turn urges the lower jaw 904B away from the upper jaw 904A (e.g., into a disengaged position).

The upper and lower jaws 904A, 904B may be further configured to hermetically seal and cut the collection chamber 104C. In one embodiment, the opposing surfaces of the jaws 904A, 904B may be heated. By placing a selected region of the collection chamber 104C (e.g., an inlet port such as 212A or exhaust port such as 212B) between the jaws 904A, 904B and compressing the jaws 904A, 904B together, opposing surfaces of the collection chamber 104C may be fused together, forming a hermetic seal. With sufficient pressure and heat applied to the selected region of the collection chamber 104C by the jaws 904A, 904B, this region may also be cut. Beneficially, by incorporating sealing mechanisms 900 into the spray drier device 102, the device 102 may cut and seal the collection chamber 104C from the spray drier assembly 104 while maintaining the sterile integrity of the assembly 104.

The process of sealing and cutting using the sealing mechanisms 900 may also be automated and controlled by the spray drier device 102. This automation may provide benefits including repeatability and reliability of the seals, reducing the possibility of contaminants entering the collection chamber 104C during the sealing and cutting process. For example, automation may ensure that the collection chamber 104 is sealed and then cut, rather than cut and sealed due to operator error.

The discussion will now turn to FIGS. 10-12, which illustrates embodiments of user workflow processes 1000, 1100 and user interfaces 1200 for use with the spray drier device 102 and spray drier assembly 104 as discussed above. It may be understood that the flow diagrams of FIGS. 10-12 may include additional elements or omit certain elements. Furthermore, the order of disclosed operations may be changed without limit.

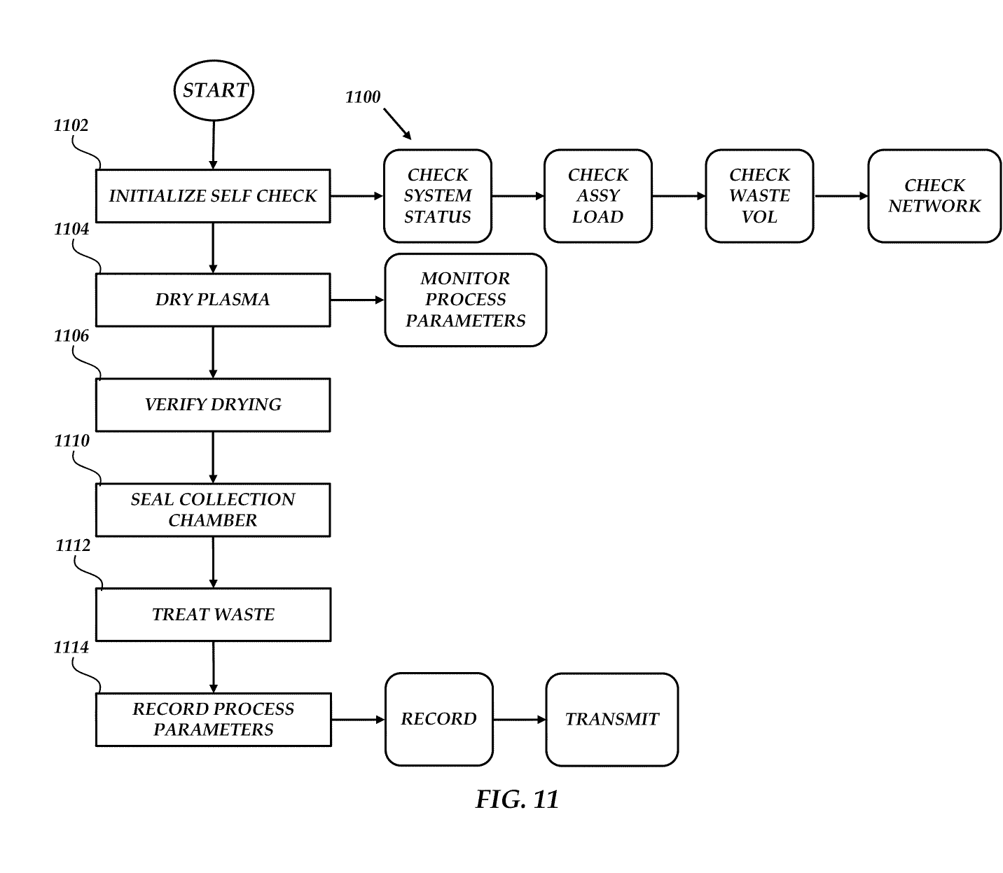

The workflow processes of FIGS. 10 and 11 will be discussed below in combination with the user interface 1200, as appropriate. In general, embodiments of the user interface 1200 may include a plurality of user interface elements 1220. The user interface elements 1220 may be further divided into function-specific groupings, such as mode 1202, result 1204, barcode 1206, and attention 1210. The user interface elements 1220 may further include a plurality of indicator lights 1222 and user selectable elements 1224. The indicator lights 1222 may direct the operator's attention to the respective user interface element 1220 for possible action. The user selectable elements 1224 may allow the operator to perform the function associated with the respective user interface element 1220.

FIG. 10 is a flow diagram illustrating an embodiment of a workflow process 1000 for tracking the liquid plasma source 112 and spray drier assembly 104 employed in a spray drying operation employing the spray drier device 102. The process 1000 may include operations of entering operator information 1002, entering plasma source information 1004, sterilely attaching the plasma source 1006, docking the spray drier assembly 1010, and performing tracking and/or quality control (QC) operations 1012.

In an embodiment, the spray drier computing device 124 may be in communication with a user interface such as an alpha-numeric keypad, a biometric scanner (e.g., fingerprint scanner, etc.), a barcode reader, an RF reader, or other input device adapted to receive information pertinent to spray drying operations (e.g., operator information, sample information, spray drier assembly information, spray drying parameters, etc.) as discussed below. The information may be maintained in a storage device in communication with the spray drier computing device 124, along with other information pertinent to the spray drying operation. The user interface may communicate with the spray drier computing device via a wired or wireless communication protocol (e.g., Bluetooth).

In operation 1002, an operator of the spray drier device 102 may provide operator information to the spray drier computing device 124. The operator information may include, but is not limited to, an operator identifier that uniquely identifies the operator using the spray drier device 102 (e.g., an operator name, an operator employee number, etc).

Beneficially, tracking the operator of the spray drying device 102 may assist with quality control. For example, should problems be identified in dried plasma resulting from the spray drier device 102, the operator information may help to determine if the problems are due systematically to certain operators or if the problems arise independently of the operator. If the problems appear to be linked to certain operators, then additional training may be appropriate for those operators to address the problems. If the problems appear to be independent of the operator, then the spray drier device 102 itself may be further investigated as the root cause of the problems.

In operations 1004, plasma source information may be provided to the spray drier computing device 124. The plasma source information may include, but is not limited to, a donation identification number (DIN), a plasma product code, a collection date/time, a plasma expiration date, and a plasma volume. The donation identification number may be an identifier which uniquely specifies a donor of the plasma. The plasma product code may be an identifier that uniquely specifies a state of the plasma (e.g., fresh-frozen plasma, pooled plasma, pooled fresh-frozen plasma, etc.). The collection date/time may specify the date of collection of the whole blood from which the plasma was obtained. The plasma expiration date may be a date beyond which the plasma cannot be used according to standard plasma handling procedures. The plasma volume may specify the volume of plasma to be dried. In certain embodiments, the plasma volume may be an integer number of standard blood units or an arbitrary amount (e.g., in the case of pooled plasma sources). As discussed below, the plasma information may be cross-checked against other information provided to the spray drier computing device 124 in order to ensure the quality of the resultant dried plasma and facilitate tracing the plasma source to the resultant dried plasma.

In operations 1006, the plasma source 112 is sterilely attached to the spray drier device 102 at the sterile dock 502. For example, a feed line from the plasma source 112 may be placed in fluid communication with pump 504A (e.g., a peristolic pump). The feed line may further be in communication with a plasma fluid detector for detecting the presence and/or flow rate of the flow of liquid sample 112A.

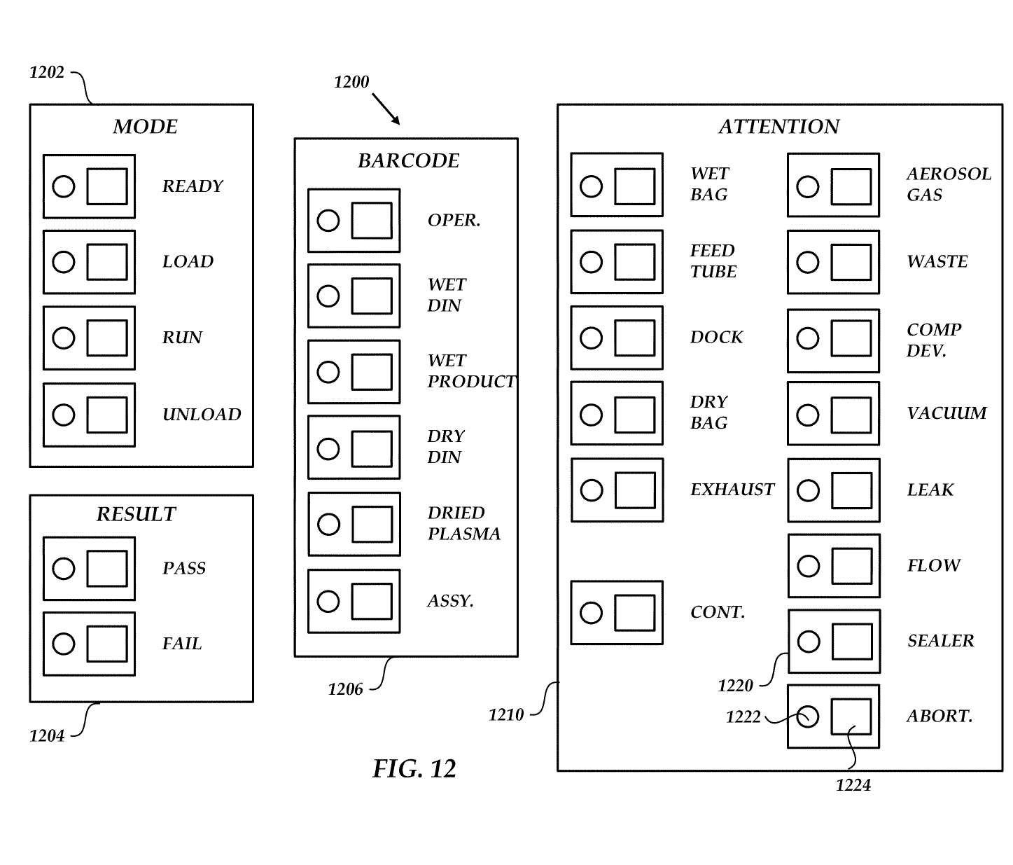

In operations 1010, the spray drier assembly 104 may be docked to the spray drier device 102 at the dock 120. The mechanical attachment and securing of the spray drier assembly 104 may be conducted as discussed above. For example, the “LOAD” interface object in grouping 1202 of FIG. 12 may allow the operator to actuate the locking mechanism 610. In addition, a plurality of spray drier assembly information may be provided to the spray drier computing device 124. The assembly information may include, but is not limited to, an assembly code, an assembly lot number, and an assembly expiration date. The assembly code may be a product code that specifies parameters of the spray drier assembly such as model (e.g., form factor), volume, and the like. The assembly lot number may be an identifier that uniquely identifies a production run during which the assembly 104 was manufactured. The assembly expiration date may be a date after which the assembly 104 is not recommended for use.

In operations 1012, a plurality of tracking and quality control operations may be performed. For example, in one aspect, a printer or other physical output device may be in communication with the spray drier computing device 124. The computing device 124 may use the printer to print out a label for the spray drier assembly that includes one or more of the operator information, the plasma information, and the assembly information. This label may be subsequently affixed to the collection chamber 104C of the spray drier assembly 104 (e.g., by the operator).

Prior to conducting spray drier operations, the label may be scanned by the operator. In response, the spray drier computing device 124 may cross-check the information contained in the label with one or more of the operator information, the plasma information, the assembly information, and/or reference values. If any discrepancies are identified in this cross-check, the spray drier computing device 124 may not authorize the spray drier device 102 to perform spray drying operations. Alternatively, if no discrepancies are identified in this cross-check, the spray drier computing device 124 may authorize the spray drier 102 to perform spray drying operations.

A discrepancy may arise when the cross-checked information does not match or does not satisfy a plurality of selected comparison criteria. For example, assume that the expiration date of the liquid sample source 112 or the spray drier assembly 104 is compared against the date of the spray drying operation. In this case, a comparison criterion may be where the expiration date falls (i.e., before or after) with respect to the date of the spray drying operation. A discrepancy may be identified if the date of the spray drying operation falls after either expiration date. A discrepancy may not be identified if the date of the spray drying operation falls before either of the respective expiration dates.

In another example, assume that the volume of the liquid sample source 112 is compared against the volume of the spray drier assembly. In this case, a selected comparison criterion would be whether the volume of the spray drier assembly is large enough to contain the volume of dried plasma produced from the volume of the liquid sample source 112. The comparison criteria could further include a scaling factor to account for the decrease in volume of the plasma during the drying process. A discrepancy may be identified if the volume of the spray drier assembly 104 is smaller than the volume of dried plasma to be produced from the volume of the liquid sample source 112. A discrepancy may not be identified if the volume of the collection chamber 104C dedicated for dried plasma storage is larger than the volume of dried plasma to be produced from the volume of the liquid sample source 112.

In a further example, assume that the DIN of the liquid sample source 112 stored by the spray drier computing device 124 is compared against the DIN recorded on the label of the spray drier collection chamber 104C. In this case, a selected comparison criterion would be whether the two DINs match. A discrepancy may be identified if the DINs do not match, while a discrepancy may not be identified if the DINS match. A similar cross-check could also be performed for the model of the spray drier assembly 104 or the operator identity.

This cross-check is beneficial because it ensures that the information affixed to the spray drier assembly 104 accurately represents the information entered to the spray drier computing device 124 regarding the operator information, the liquid sample information, and the assembly information. For example, consider the case where an operator is conducting spray drying operations on a large number of liquid samples. Under these circumstances, the operator may handle many liquid samples and spray drier assemblies 104, and the operator may easily identify the wrong liquid sample information on the collection chamber 104C inadvertently. Once such an error is made, it may be difficult or impossible to identify and/or correct this error.

However, by checking the sample information on the collection chamber 104C with the sample information stored by the spray drying computing device 124 for the spray drying operation, such errors may be substantially reduced or eliminated. In an example, discrepancies or lack thereof may be identified to the operator in the user interface 1200 (e.g., the “barcode” section 1206 may alert the operator to discrepancies identified in the cross-check).

For example, the “OPER” user interface object represents identification of the operator. The operator information may be cross-checked against a list of approved operators (e.g., operators who have been trained in use of the spray drier device 102) maintained by the computing device 124. If the operator's name is contained within the list of approved operators, the computing device 124 may authorize the spray drier device 102 for use. If the operator's name is not contained within the list of approved operators, the computing device may not authorize the spray drier device 102 to perform spray drying operations.

The “Wet DIN” user interface object refers to the DIN of the plasma source to be dried. The “Wet Product” user interface object refers to the product code of the plasma source to be dried. The “Dry DIN” user interface object refers to the DIN of the dried plasma to be collected. The “Dried Plasma” user interface object refers to the product code of the dried plasma to be collected. The “ASSY” user interface object refers to the product code of the spray drier assembly 104 used in the spray drying operation. The computing device 124 may cross-check any one or more of the values of the DIN of the plasma source to be dried, the product code of the plasma source to be dried, the DIN of the dried plasma to be collected, the product code of the dried plasma to be collected with the corresponding value maintained by the computing device 124. A discrepancy may be identified if the cross-check identifies a failure to match and the respective indicator 1220 for discrepancy (e.g., a red light) may be shown in the user interface 1200. A discrepancy may not be identified if the cross-check identifies a match and the respective indicator 1220 for a match (e.g., a green light) may be shown in the user interface 1200. Beneficially, with knowledge of any discrepancies, the operator may diagnose and remedy the source of the discrepancy. As a result, the ability to track the liquid sample source 112 once it is spray dried is significantly improved.

With further reference to FIG. 11, a flow diagram is provided that illustrates an embodiment of a workflow process 1100 for monitoring spray drying parameters and transmitting spray drying information to a remote computing device (e.g., computing device 150). Once the spray drier device 102 is ready to perform spray drying operations (e.g., after the operations discussed with respect to FIG. 10 are completed), the user interface may indicate this state of readiness on the user interface 1200 (e.g., the “READY” user interface element within the “MODE” grouping 1202). Selection of the “RUN” user interface element may start the spray drying process.

In an embodiment, the spray drier device 102 may perform self-check operations 1102 after the “RUN” user interface object is selected and prior to beginning spray drying operations. The self-check may include, but is not limited to, checking system status, checking the spray drier assembly 104, checking waste volume, and checking network status. The system status check may confirm that one or more of the following are operating correctly: the flow of aerosolizing gas 114A, the flow of drying gas 116A, power distribution, and that the cover 800 is closed. The self-check may further check that the liquid sample source 112 is present (e.g., a scale that measures the weight of the liquid sample, flow of the liquid sample 112A, etc.). The self-check may further determine that the locking mechanism is locked. The self-check may further determine that the collection chamber that receives waster 122 has sufficient capacity for receiving additional waste water 122 in the current spray drying operation. The self-check may also determine that the spray drier computing device 124 is in communication with the remote computing device 150.

Provided that the self-check is completed without error, the process flow 1100 moves to operations 1104, where spray drying parameters are monitored. Examples of the spray drying parameters may include, but are not limited to, flow rates and temperatures of the respective flows 112A, 114, and 116A.

After the sample source 112 is exhausted, the computing device 124 may verify the completion of the drying process in operations 1106. In this operation, the spray computing device 124 compares the measured spray drying processing parameters (e.g., flow rates, temperatures, times) from a liquid sample dried in an idealized process run. A spray drying operation for which the measured processing parameters fall within a pre-determined range of the ideal processing parameters is approved. A spray drying operation for which the measured processing parameters fall outside a pre-determined range of the ideal processing parameters is not approved. Subsequently, in operations 1110, the collection chamber 104C may be sealed and detached from the assembly 104C as discussed above. The waste 122 may be further treated in operations 1112.

In operations 1114, the information collected by the spray drier computing device 124 may be transmitted to remote computing device 150. This information may include, but is not limited to, the operator information, the sample information, the assembly information, and the spray drying parameters. If the remote computing device 150 is unavailable, the computing device 124 may further store the collected information until such time as communication with the remote computing device 150 is re-established.

In the event of any problems or other matters requiring an operator's attention during spray drying operations, the user interface 1200 may be updated. For example, as illustrated in the “ATTENTION” grouping 1210, user interface elements may be provided for problems associated with the assembly 104 (e.g., the “Wet Bag,” “Dry Bag” user interface objects), the flow of liquid sample 112 (the “Feed Tube” user interface object), the dock 120 (the “Dock” user interface object), exhaust of the humid air 208 (the “Exhaust” user interface object), the flow of aerosol gas 114A (the “Aerosol Gas” user interface object), the waste 122 (the “Waste” user interface object), the computing device 124 (“Comp.Dev.” user interface object), vacuum (the “Vacuum” user interface object), any loss of pressure (the “Leak” user interface object), the flow rates of aerosolizing gas 114A, the flow of drying gas 116A, or flow of plasma 112A is outside of a selected range (the “Flow” user interface object), and the sealing device 900 (the “Sealer” user interface object), amongst others. The operator, informed of a problem, may diagnose and remedy it. The operator may select the “Continue” user interface element to reflect that a problem has been addressed. Alternatively, if the problem cannot be addressed and spray drying operations are to be terminated, the operator may select the “Abort” user interface object. Upon completion of a successful spray drying operation (e.g., a drying operation that is verified and run to completion without aborting), the user interface may reflect a result of “Pass,” while a drying operation that is not verified or terminated early may reflect a result of “Fail.” Any problems identified during spray drying operations and the Pass or Fail result may also be recorded in the information collected and transmitted by the spray drier computing device 124.

In certain embodiments, spray drier system 100 may be housed in a blood center and the remote computing device 150 may be part of an inventory tracking system that maintains information regarding stored liquid plasma (e.g., FFP) as well as dried plasma obtained from spray drying operations. As such, the remote computing system 150 may already maintain a data structure (e.g., a database) that includes at least a portion of the sample information. When the computing system 124 transmits the information collected by the spray drier device 102 to the remote computing system 150, the remote computing system 150 may match the sample information contained within the transmitted, collected spray drier information to the sample information it already maintains. The remote computing system 150 may further update the data structure to include the collected spray drier information.

Beneficially, by providing the computing device 124 in communication with the remote computing device 150, sample tracking, from collection through use, may be conveniently maintained by a blood back. The blood back may maintain original records regarding a plasma sample while stored in its liquid state (e.g., FFP). Once the plasma sample is spray dried, the computing system 124 may transmit the collected spray drier information to the blood bank (e.g., remote computing device 150), where the blood bank's sample records are updated. Subsequently, when the dried plasma is used, the blood bank possesses a complete and accurate tracking history of the plasma sample which is readily accessible from the data structure by the remote computing device 150.

The above-described systems and methods can be implemented in digital electronic circuitry, in computer hardware, firmware, and/or software. The implementation can be as a computer program product. The implementation can, for example, be in a machine-readable storage device, for execution by, or to control the operation of, data processing apparatus. The implementation can, for example, be a programmable processor, a computer, and/or multiple computers.

A computer program can be written in any form of programming language, including compiled and/or interpreted languages, and the computer program can be deployed in any form, including as a stand-alone program or as a subroutine, element, and/or other unit suitable for use in a computing environment. A computer program can be deployed to be executed on one computer or on multiple computers at one site.

Method steps can be performed by one or more programmable processors executing a computer program to perform functions of the invention by operating on input data and generating output. Method steps can also be performed by and an apparatus can be implemented as special purpose logic circuitry. The circuitry can, for example, be a FPGA (field programmable gate array) and/or an ASIC (application-specific integrated circuit). Subroutines and software agents can refer to portions of the computer program, the processor, the special circuitry, software, and/or hardware that implement that functionality.

Processors suitable for the execution of a computer program include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer can include, can be operatively coupled to receive data from and/or transfer data to one or more mass storage devices for storing data (e.g., magnetic, magneto-optical disks, or optical disks).

Data transmission and instructions can also occur over a communications network. Information carriers suitable for embodying computer program instructions and data include all forms of non-volatile memory, including by way of example semiconductor memory devices. The information carriers can, for example, be EPROM, EEPROM, flash memory devices, magnetic disks, internal hard disks, removable disks, magneto-optical disks, CD-ROM, and/or DVD-ROM disks. The processor and the memory can be supplemented by, and/or incorporated in special purpose logic circuitry.

To provide for interaction with a user, the above described techniques can be implemented on a computer having a display device. The display device can, for example, be a cathode ray tube (CRT) and/or a liquid crystal display (LCD) monitor. The interaction with a user can, for example, be a display of information to the user and a keyboard and a pointing device (e.g., a mouse or a trackball) by which the user can provide input to the computer (e.g., interact with a user interface element). Other kinds of devices can be used to provide for interaction with a user. Other devices can, for example, be feedback provided to the user in any form of sensory feedback (e.g., visual feedback, auditory feedback, or tactile feedback). Input from the user can, for example, be received in any form, including acoustic, speech, and/or tactile input.

The above described techniques can be implemented in a distributed computing system that includes a back-end component. The back-end component can, for example, be a data server, a middleware component, and/or an application server. The above described techniques can be implemented in a distributing computing system that includes a front-end component. The front-end component can, for example, be a client computer having a graphical user interface, a Web browser through which a user can interact with an example implementation, and/or other graphical user interfaces for a transmitting device. The components of the system can be interconnected by any form or medium of digital data communication (e.g., a communication network). Examples of communication networks include a local area network (LAN), a wide area network (WAN), the Internet, wired networks, and/or wireless networks.

The system can include clients and servers. A client and a server are generally remote from each other and typically interact through a communication network. The relationship of client and server arises by virtue of computer programs running on the respective computers and having a client-server relationship to each other.

Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), 802.11 network, 802.16 network, general packet radio service (GPRS) network, HiperLAN), and/or other packet-based networks. Circuit-based networks can include, for example, the public switched telephone network (PSTN), a private branch exchange (PBX), a wireless network (e.g., RAN, bluetooth, code-division multiple access (CDMA) network, time division multiple access (TDMA) network, global system for mobile communications (GSM) network), and/or other circuit-based networks.

The transmitting device can include, for example, a computer, a computer with a browser device, a telephone, an IP phone, a mobile device (e.g., cellular phone, personal digital assistant (PDA) device, laptop computer, electronic mail device), and/or other communication devices. The browser device includes, for example, a computer (e.g., desktop computer, laptop computer) with a world wide web browser (e.g., Microsoft®Internet Explorer® available from Microsoft Corporation, Mozilla® Firefox available from Mozilla Corporation). The mobile computing device includes, for example, a Blackberry®.

The terms comprise, include, and/or plural forms of each are open ended and include the listed parts and can include additional parts that are not listed. And/or is open ended and includes one or more of the listed parts and combinations of the listed parts.

One skilled in the art will realize the technology may be embodied in other specific forms without departing from the spirit or essential characteristics thereof. The foregoing embodiments are therefore to be considered in all respects illustrative rather than limiting of the technology described herein. Scope of the technology is thus indicated by the appended claims, rather than by the foregoing description, and all changes that come within the meaning and range of equivalency of the claims are therefore intended to be embraced therein.

What is claimed is:

1. A tracking system for a spray drier device, the tracking system comprising:
a first computing device in communication with a spray drier device, the first computing device adapted to receive:
operator information regarding an operator of a spray drier device;
sample information regarding a liquid sample to be spray dried by the spray drier device;
assembly information regarding a spray drier assembly adapted to couple with the spray drier device for spray drying the liquid sample; and
operating parameters for a spray drying operation performed by the spray device to dry the liquid sample;
a user interface in communication with the first computing device, the user interface adapted to display at least one status associated with one or more of the operator information, the sample information, the assembly information, and the operating parameters;
wherein the first computing device is further adapted to output at least one of the operator information, the sample information, the assembly information, and the operating parameters to a second computing device maintaining a record of the sample information.

2. The tracking system of claim 1, wherein the spray drier device comprises:
a liquid sample port for receiving a flow of a liquid sample; and
a spray drier device dock adapted to couple with the spray drier assembly positioned within the dock, the dock including:
an aerosolizing gas port for receiving the flow of an aerosolizing gas; and
a dryer gas port for receiving the flow of drying gas;
wherein the aerosolizing gas port is not co-axial with the dryer gas port.

3. The tracking system of claim 2, wherein the spray drier device further comprises:
a locking mechanism positioned adjacent to the dock and configured to couple with a spray drier assembly positioned within the dock; and
an actuator configured to move the locking mechanism between an engaged and a disengaged position, wherein the locking mechanism inhibits removal of the spray drier assembly from the dock in the engaged position and wherein the locking mechanism does not inhibit removal of the spray drier assembly from the dock in the disengaged position.

4. The tracking system of claim 1, wherein the operator information comprises an identity of the operator.

5. The tracking system of claim 1, wherein the sample information comprises one or more of a donor identification number (DIN), a sample collection date, a sample volume, and a sample expiration date.

6. The tracking system of claim 1, wherein the assembly information comprises one or more of an assembly product code, an assembly lot number, and an assembly expiration date.

7. The tracking system of claim 1, wherein the operating parameters comprise one or more of flow rate and temperature for one or more of the flows of liquid sample, aerosolizing gas, and drying gas.

8. The tracking system of claim 2, wherein the spray drier device further comprises a plurality of heaters adapted to heat the spray drier assembly by electromagnetic radiation and wherein the operating parameters comprise output of the plurality of heaters.

9. The tracking system of claim 1, wherein the first computing device is adapted to receive one or more of the operator information, the sample information, and the assembly information by bar code.

10. The tracking system of claim 1, wherein the first computing device is further adapted to:
compare the operating parameters to a plurality of quality control criteria; and
determining a quality of the spray drying operation based upon the comparison.

11. The tracking system of claim 2, wherein the first computing device is adapted for remote access by an operator for performing one or more of monitoring the operating parameters during the spray drying operation, reviewing spray drier device alerts, and performing diagnostics on the spray drier device.

12. The tracking system of claim 1, wherein the first computing device is in communication with at least two spray drier devices and wherein the first computing device is adapted to receive a plurality of status indications from each of the spray drier devices and display the respective status indications for each of the spray drier devices on a common display device.

13. The tracking system of claim 2, wherein the first computing device is in communication with a data store, the data store adapted to maintain operating parameters for a plurality of spray drying operations performed by the spray drier device.

14. A method for tracking a liquid sample undergoing spray drying, the method comprising:
receiving, by a first computing device in communication with a spray drier device:
operator information regarding an operator of a spray drier device;
sample information regarding a liquid sample to be spray dried by the spray drier device;
assembly information regarding a spray drier assembly adapted to couple with the spray drier device for spray drying the liquid sample; and
operating parameters for a spray drying operation performed by the spray device to dry the liquid sample;
displaying, by a user interface in communication with the first computing device, at least one status associated with one or more of the operator information, the sample information, the assembly information, and the operating parameters; and
outputting, by the first computing device, at least one of the operator information, the sample information, the assembly information, and the operating parameters to a second computing device maintaining a record of the sample information.

15. The method of claim 14, wherein the spray drier device comprises:
a liquid sample port for receiving the flow of a liquid sample; and
a spray drier device dock adapted to couple with the spray drier assembly positioned within the dock, the dock including:
an aerosolizing gas port for receiving the flow of an aerosolizing gas; and
a dryer gas port for receiving the flow of drying gas;
wherein the aerosolizing gas port is not co-axial with the dryer gas port.

16. The method of claim 15, wherein the spray drier device further comprises:
a locking mechanism positioned adjacent to the dock and configured to couple with a spray drier assembly positioned within the dock; and
an actuator configured to move the locking mechanism between an engaged and a disengaged position, wherein the locking mechanism inhibits removal of the spray drier assembly from the dock in the engaged position and wherein the locking mechanism does not inhibit removal of the spray drier assembly from the dock in the disengaged position.

17. The method of claim 15, wherein the operator information comprises an identity of the operator.

18. The method of claim 15, wherein the sample information comprises one or more of a donor identification number (DIN), a sample collection date, a sample volume, and a sample expiration date.

19. The method of claim 15, wherein the assembly information comprises one or more of an assembly product code, an assembly lot number, and an assembly expiration date.

20. The method of claim 15, wherein the operating parameters comprise one or more of flow rate and temperature for one or more of the flows of liquid sample, aerosolizing gas, and drying gas.

21. The method of claim 10, wherein the spray drier device further comprises a plurality of heaters adapted to heat the spray drier assembly by electromagnetic radiation and wherein the operating parameters comprise output of the plurality of heaters.

22. The method of claim 15, further comprising:
comparing the operating parameters to a plurality of quality control criteria; and
determining a quality of the spray drying operation based upon the comparison.

23. The method of claim 15, further comprising providing, by the first computing device, remote access to the spray drier device by an operator of the spray drier device for performing one or more of monitoring the operating parameters during the spray drying operation, reviewing spray drier device alerts, and performing diagnostics on the spray drier device

24. The method of claim 15, further comprising:
receiving, by the first computing device, a plurality of status indications from each of the spray drier devices; and
displaying, by the first computing device, the respective status indications for each of the spray drier devices on a common display device.

25. The method of claim 15, further comprising communicating, by the first computing device, with a data store, the data store adapted to maintain operating parameters for a plurality of spray drying operations performed by the spray drier device.

26. The method of claim 15, wherein the first computing device is adapted to receive one or more of the operator information, the sample information, and the assembly information by bar code.

27. A method of controlling a spray drying operation, the method comprising:
receiving, by a first computing device:
sample information regarding a liquid sample to be spray dried by a spray drier device;
assembly information regarding a spray drier assembly adapted to couple with the spray drier device for spray drying the liquid sample;
comparing, by the first computing device, each of the sample information and the assembly information to a plurality of respective reference values;
authorizing, by the first computing device, the spray drier device to perform a spray drying operation to spray dry the liquid sample for liquid sample information and assembly information satisfying their respective plurality of reference values.

28. The control method of claim 27, further comprising not authorizing the spray drier device to spray dry the liquid sample for either liquid sample information or assembly information failing to satisfy their respective plurality of reference values.

29. The control method of claim 27, wherein the liquid sample information and the spray drier assembly information each comprise respective expiration dates and the respective reference values each comprise a current date for spray drying operations, wherein the liquid sample information and the spray drier assembly information each satisfy their respective reference value for current dates prior to the respective expiration dates.

30. The control method of claim 27, wherein the liquid sample information comprises a volume of the liquid sample and the plurality of reference values for the liquid sample comprises a volume of the spray drier assembly and wherein the liquid sample information satisfies the plurality of reference values for the liquid sample for liquid sample volumes less than the spray drier assembly volume.

31. The control method of claim 27, wherein comparing each of the sample information and the assembly information to a plurality of respective reference values further comprises:
printing, by a printing device in communication with the first computing device, a bar code including the sample information;
affixing the bar code including the sample information and the assembly information to the spray drier assembly; and
comparing the sample information and the assembly information included in the bar code to the plurality of respective reference values.

32. The control method of claim 27, further comprising outputting, by the first computing device, at least one of the operator information, the sample information, the assembly information, and operating parameters for the authorized spray drying operation to a second computing device maintaining a record of the sample information
